(19) United States
(12) Patent Application Publication
(10) Pub. No.: US 2008/0255150 A1
(43) Pub. Date: Oct. 16, 2008

(54) Novel Compounds

(75) Inventor: Timothy Luker, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/092,431

(22) PCT Filed: Nov. 1, 2006
(86) PCT No.: PCT/GB06/04075

§ 371 Date: May 2, 2008

(30) Foreign Application Priority Data
Nov. 5, 2005 (GB) 0522619.6
Apr. 12, 2006 (GB) 0607353.0

(51) Int. Cl. A61K 31/495; C07D 241/04; A61P 11/00

(52) U.S. Cl. 514/255.01; 544/384; 544/391; 514/255.02

(57) ABSTRACT

The invention relates to substituted aryl acids as useful pharmaceutical compounds for treating respiratory disorders, pharmaceutical compositions containing them, and processes for their preparation.

The present invention relates to substituted aryl acids as useful pharmaceutical compounds for treating respiratory disorders, pharmaceutical compositions containing them, and processes for their preparation.

EPA 1 170 594 discloses methods for the identification of compounds useful for the treatment of disease states mediated by prostaglandin D2, a ligand for orphan receptor CRTH2. GB 1356834 discloses a series of compounds said to possess anti-inflammatory, analgesic and antipyretic activity. It has been found that certain phenoxyacetic acids are active at the CRTH2 receptor, and as a consequence are expected to be potentially useful for the treatment of various respiratory diseases, including asthma and COPD.
In a first aspect the invention therefore provides compound of formula (I) or a carboxylic acid bioisostere thereof:

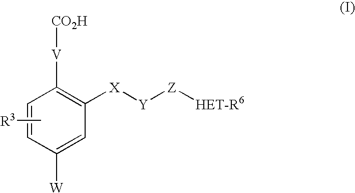

in which:

V is CR¹R², CR¹R²—CR¹R² or V is S(O)ₙCR¹R² (where n is 0, 1 or 2), NR¹¹CR¹R², CCR¹R², CR¹R²C or CR¹CR²;

R¹ and R² independently represent a hydrogen atom, halogen, C₂-C₆ alkenyl, C₂-C₆ alkynyl, C₃-C₇ cycloalkyl or a C₁₋₆alkyl group, the latter four groups being optionally substituted by one or more substituents independently selected from halogen, C₃-C₇ cycloalkyl, NR⁹R¹⁰, OR⁸, S(O)ₙR⁷ (where n is 0, 1 or 2);
or
R¹ and R² together can form a 3-8 membered ring optionally containing one or more atoms selected from O, S, NR¹¹ and itself optionally substituted by one or more C₁-C₃ allyl or halogen;
W is hydrogen, halogen, cyano, nitro, SO₂R⁷, SO₂NR⁹R¹⁰, OR⁸, or C₁₋₆alkyl, the latter being optionally substituted by one or more substituents independently selected from halogen, OR⁸ and NR⁷R⁸, S(O)ₙR⁵ where n is 0, 1 or 2.

R³ is one or more substituents independently selected from hydrogen, halogen, CN, nitro, SO₂R⁷, OR⁸, SR⁷, SOR⁷, SO₂NR⁹R¹⁰, CONR⁹R¹⁰, NR⁹R¹⁰, NR¹¹SO₂R⁷, NR¹¹CO₂R⁷, NR¹¹COR⁷ or C₁₋₆alkyl, the latter being optionally substituted by one or more substituents independently selected from halogen, OR⁸ and NR⁹R¹⁰, S(O)ₙR⁷ where n is 0, 1 or 2;

X represents a bond, or C₁-C₆ alkyl, optionally substituted by one or more substituents independently selected from halogen, C₁-C₆ alkyl the latter being optionally substituted by one or more substituents independently selected from halogen, OR⁶ and NR⁷R⁸, S(O)ₙR⁵ where n is 0, 1 or 2;

Y represents a diamine of the following type:—

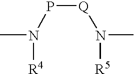

R⁴ and R⁵ independently represent hydrogen, SO₂R⁷, C(O)R⁷, CO₂R⁷ and C₁-C₆ alkyl, the latter being optionally substituted by one or more substituents independently selected from aryl, heteroaryl, halogen, OR⁸ and NR⁹R¹⁰, S(O)ₙR⁷ where n is 0, 1 or 2;
R⁴ and R⁵ are joined together or one of R⁴ and R⁵ is joined onto P or Q to form a saturated heterocyclic 3-10 membered ring with, 1 or 2 endocyclic nitrogen atoms;

P and Q independently represent, C₁-C₆ alkyl optionally substituted by one or more substituents independently selected from (═O), halogen, OR⁸ and NR⁹R¹⁰, S(O)ₙR⁷ (where n is 0, 1 or 2), C₁-C₆ alkyl, C₃-C₆ cycloalkyl, aryl or heteroaryl (the latter two being optionally substituted by one or more substituents independently selected from halogen, OR⁸ and NR⁹R¹⁰, CONR⁹R¹⁰, S(O)ₙR⁷ where n is 0, 1 or 2);

Z represents a bond, (CR¹²)n-C(O), (CR¹²)n-S(O)n, C(O)(CR¹²)n, or S(O)₂(CR¹²)n, S(O)₂N(CR¹²)n, where n=0, 1 or 2;

HET represents aryl or heteroaryl;
R⁶ represents one or more substituents independently selected from hydrogen, halogen, CN, nitro, COR⁷, CO₂R⁸, SO₂R⁷, OR⁸, SR⁸, SOR⁷, SO₂NR⁹R¹⁰, CONR⁹R¹⁰, NR⁹R¹⁰, NR⁸SO₂R⁷, NR⁸CO₂R⁸, NR⁸COR⁷, NR⁸CONR⁹R¹⁰, NR⁸SO₂NR⁹R¹⁰, aryl, heteroaryl, C₂-C₆ alkenyl, C₂-C₆ alkynyl, C₃-C₇ cycloalkyl or C₁₋₆alkyl, the latter four groups being optionally substituted by one or more substituents independently selected from halogen, C₃-C₇ cycloalkyl, CN, OR⁸, NR⁹R¹⁰, S(O)ₙR⁷ (where n is 0, 1 or 2), CONR⁹R¹⁰, NR⁸COR⁷, SO₂NR⁹R¹⁰ and NR⁸SO₂R⁷;

R⁷ represents a C₁-C₆ alkyl, an aryl or a heteroaryl group all of which may be optionally substituted by halogen atoms, OR⁸, NR¹⁴R¹⁵;

R⁸ represents hydrogen, C₁-C₆, alkyl, an aryl or a heteroaryl group all of which may be optionally substituted by halogen atoms, OR⁸, NR¹⁴R¹⁵;

R⁹ and R¹⁰ independently represent hydrogen, C₃-C₇ cycloalkyl or C₁₋₆alkyl, the latter two groups being optionally substituted by one or more substituents independently selected from halogen, C₃-C₇ cycloalkyl, OR⁶ and NR¹⁴R¹⁵, S(O)ₙR⁶ (where n=0, 1 or 2), CONR⁷R⁸, NR⁶COR⁷, SO₂NR⁷R⁸ and NR⁶SO₂R⁵;

or

R⁹ and R¹⁰ together with the nitrogen atom to which they are attached can form a 3-8 membered saturated heterocylic ring optionally containing one or more atoms selected from O, S(O)ₙ (where n=0, 1 or 2), NR¹³, and itself optionally substituted by halogen or C₁₋₃ alkyl;

R¹¹ represents a hydrogen atom, C(O)R⁹, C₁-C₆ alkyl an aryl or a heteroaryl group (the latter three can be optionally substituted by halogen);

R¹² represents one or more from hydrogen, or a C₁₋₆alkyl group, the latter being optionally substituted by one or more substituents independently selected from halogen, C₃-C₇ cycloalkyl, NR¹⁴R¹⁵, OR⁸, S(O)ₙR⁷ (where n is 0, 1 or 2);

R¹³ represent hydrogen, C₁₋₄ alkyl, —COC₁-C₄ alkyl, COYC₁-C₄alkyl where Y is O or NR⁷; and

R¹⁴ and R¹⁵ independently represent hydrogen, C₁₋₄ alkyl

or

R¹⁴ and R¹⁵ together with the nitrogen atom to which they are attached can form a 3-8 membered saturated heterocylic ring optionally containing one or more atoms selected from O, S(O)ₙ (where n=0, 1 or 2), NR¹³, and itself optionally substituted by halogen or C₁₋₃ alkyl;

and pharmaceutically acceptable salts thereof.

It is to be understood that the group —CO₂H, as used herein, includes carboxylic acid bioisosteres. This is a term familiar to medicinal chemists and refers to functional groups which have similar acid-base characteristics to a carboxylic acid group. Well known carboxylic acid isosteres include, but are not limited to, the following groups:

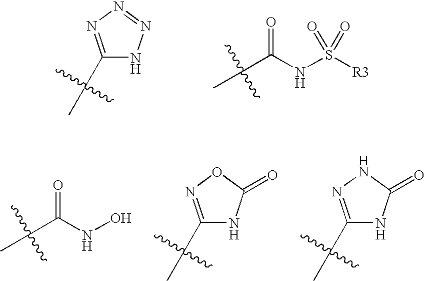

Examples of monocyclic saturated rings as defined for Y include piperazine, alkyl substituted piperazine (such as methyl, ethyl or propyl piperazine), piperazinone, imidazolidine, homopiperazine, aminopyrrolidine, aminoazetidine and aminopiperidine.

Examples of aryl include phenyl and naphthyl.

Heteroaryl is defined as a 5-7 member aromatic ring or can be 6,6- or 6,5-fused bicyclic ring optionally containing one or more heteroatoms selected from N, S and O. The bicyclic ring may be linked through carbon or nitrogen and may be attached through the 5 or 6 membered ring and can be fully or partially saturated. Examples include pyridine, pyrimidine, thiazole, oxazole, pyrazole, imidazole, furan, isoxazole, pyrrole, isothiazole and azulene, naphthyl, indene, quinoline, isoquinoline, indole, indolizine, benzo[b]furan, benzo[b]thiophene, 1H-indazole, benzimidazole, benzthiazole, benzoxazole, purine, 4H-quinolizine, cinnoline, phthalazine, quinazoline, quinoxaline, 1,8-naphthyridine, pteridine, quinolone and 1,2-methylenedioxy benzene.

In the context of the present specification, unless otherwise indicated the groups aryl and heteroaryl can be optionally substituted by R⁶.

In the context of the present specification, unless otherwise indicated, an alkyl or alkenyl group or an alkyl or alkenyl moiety in a substituent group may be linear or branched.

Heterocyclic rings as defined for R¹⁴ and R¹⁵ means saturated heterocycles, examples include morpholine, thiomorpholine, azetidine, imidazolidine, pyrrolidine, piperidine and piperazine.

Preferably V is CR¹R², CR¹R²—CR¹R², CCR¹R² or CR¹R²C, more preferably V is CH₂ or CH₂CH₂.

Preferably W is hydrogen or halogen, more preferably W is halogen, most preferably chloro.

Preferably R¹ and R² are independently hydrogen.

Preferably R³ is hydrogen.

Preferably X is CH₂.

Preferably the group Y (together with the two nitrogen atoms to which it is attached) is piperazine, which can be optionally substituted by C₁₋₄ alkyl.

Preferably the group Z is SO₂, SO₂CH₂, C(O)CH₂, more preferably SO₂CH₂ or C(O)CH₂.

Preferably HET is aryl, or heteroaryl, more preferably HET is phenyl.
Preferably R⁶ is hydrogen or one or more substituents selected from halogen, hydrogen, C₁-C₆ alkyl (optionally substituted by one or more halogen atoms), alkoxy (alkyl group is optionally substituted by halogen atoms). More preferably R⁶ is one of the substituents exemplified herein.
Preferred compounds of the invention include:
Sodium 3-(2-{[4-(benzylsulfonyl)piperazin-1-yl]methyl}-4-chlorophenyl) propanoate;
3-(2-{[(3S)-4-(benzylsulfonyl)-3-methylpiperazin-1-yl]methyl}-4-chlorophenyl)propanoic acid;
Sodium 3-(4-chloro-2-{[(3S)-3-methyl-4-(phenylsulfonyl)piperazin-1-yl]methyl}phenyl)propanoate;
3-(4-chloro-2-{[(3S)-3-methyl-4-(phenylacetyl)piperazin-1-yl]methyl}phenyl)propanoic acid;
3-[4-chloro-2-({(3S)-3-methyl-4-[(4-methylbenzyl)sulfonyl]piperazin-1-yl}methyl)phenyl]propanoic acid;
3-[4-chloro-2-({(3S)-3-methyl-4-[(3-methylbenzyl)sulfonyl]piperazin-1-yl}methyl)phenyl]propanoic acid;
3-[4-chloro-2-({(3S)-3-methyl-4-[(2-methylbenzyl)sulfonyl]piperazin-1-yl}methyl)phenyl]propanoic acid;
(2-{[(3S)-3-methyl-4-(phenylsulfonyl)piperazin-1-yl]methyl}phenyl)acetic acid;
(4-chloro-2-{[(3S)-3-methyl-4-(phenylsulfonyl)piperazin-1-yl]methyl}phenyl)acetic acid;
{4-chloro-2-[((3S)-3-methyl-4-{[4-(trifluoromethyl)phenyl]acetyl}piperazin-1-yl)methyl]phenyl}acetic acid;
[4-chloro-2-({(3S)-4-[(4-methoxyphenyl)acetyl]-3-methylpiperazin-1-yl}methyl)phenyl]acetic acid;
[4-chloro-2-({(3S)-4-[(2,4-difluorophenyl)acetyl]-3-methylpiperazin-1-yl}methyl)phenyl]acetic acid;
[4-chloro-2-({(3S)-4-[(3,4-difluorophenyl)acetyl]-3-methylpiperazin-1-yl}methyl)phenyl]acetic acid;
(2-{[(3S)-4-(benzylsulfonyl)-3-methylpiperazin-1-yl]methyl}-4-chlorophenyl)acetic acid;
[4-chloro-2-({(3S)-4-[(4-chlorophenyl)acetyl]-3-methylpiperazin-1-yl}methyl)phenyl]acetic acid;
(4-chloro-2-{[(3S)-3-methyl-4-(phenylacetyl)piperazin-1-yl]methyl}phenyl)acetic acid;
[4-chloro-2-({(3S)-4-[(4-fluorophenyl)acetyl]-3-methylpiperazin-1-yl}methyl)phenyl]acetic acid;
[4-chloro-2-({(3S)-3-ethyl-4-[(4-fluorophenyl)acetyl]piperazin-1-yl}methyl)phenyl]acetic acid;
[4-chloro-2-({(3S)-4-[(4-chlorophenyl)acetyl]-3-ethylpiperazin-1-yl}methyl)phenyl]acetic acid;
2-(2-{[(3S)-4-(benzylsulfonyl)-3-methylpiperazin-1-yl]methyl}-4-chlorophenyl)-N-(methylsulfonyl)acetamide
   and pharmaceutically acceptable salts thereof.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

The compound of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate.

It will be appreciated by those skilled in the art that in the processes of the present invention certain functional groups in the starting reagents or intermediate compound may need to be protected by protecting groups. Thus, the preparation of the compound of formula (I) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

Compounds of formula (I) can be prepared by hydrolysis of a compound of formula (II):

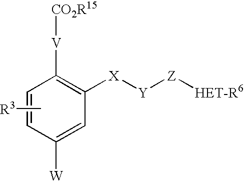

### (II)

in which R¹⁵ is methyl, ethyl or tetriary butyl, and can be removed under acidic or basic conditions for example by stirring in trifluoroacetic acid or dilute sodium hydroxide in a suitable solvent such as dichloromethane, THF or methanol. R¹, R², R³, R⁶, W, X, Y and Z are as defined in compounds of formula (I) or protected derivatives thereof. Compounds of formula (II) are novel and form an additional part of the invention.

Compounds of formula (II) are prepared from compounds of formula (III) as described in Scheme 1.

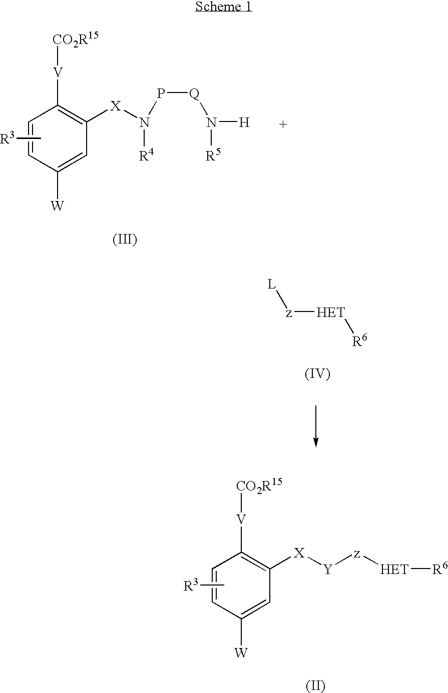

in which R¹, R², R³, R⁴, R⁵, R⁶, R¹⁵, P, Q, W, X, Y and Z are as defined in compounds of formula (II) or protected derivatives thereof.

When Z is SO₂, or C(O) the compounds of formula (III) are reacted with sulfonyl chlorides or acid chlorides of formula (IV) in which L=Chlorine. The reaction is carried out in in the presence of a base such as triethylamine, aqueous sodium hydrogen carbonate or potassium carbonate in a suitable organic solvent such as dichloromethane. When Z is alkyl compounds of formula (III) are reacted with alkyl chlorides using a suitable base such as triethylamine or sodium hydride in an organic solvent such as DMF or DCM.

When L=OH and Z=C(O) the reaction is carried out using a coupling reagent such as HATU in a suitable organic solvent such as DMF, DCM or NMP.

Compounds of formula (IV) are commercially available or can be prepared readily by those skilled in the art.

Compounds of formula (III) can be prepared from compounds of formula (V) by reacting with a diamine compound of formula (VI), by a coupling reaction in a suitable organic solvent for example THF, DMF or dichloromethane in the presence of a base such as triethylamine, potassium carbonate or the like;

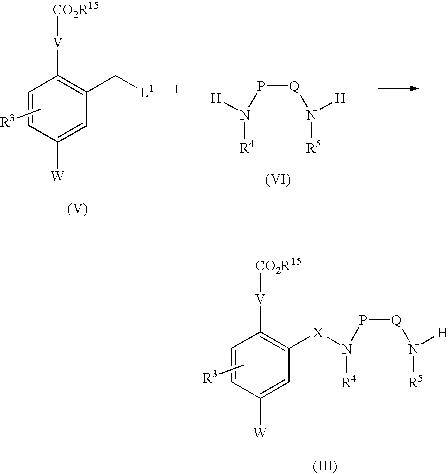

in which R¹, R², R³, R⁴, R⁵, R¹⁵, P, Q, V, W, and X are as defined in compounds of formula (II) or protected derivatives thereof. L¹ is a suitable leaving group such as mesylate or halogen.

The diamine compound of formula (VI) is monoprotected as compounds of formula (VIa) with a suitable amine protecting group such as BOC (tert-butyl carbonyl). This protecting group is subsequently removed under acidic conditions, for example TFA.

Compounds of formula (VIa) where the amine is monoprotected with the BOC protecting group are commercially available or may be protected by reacting compounds of formula (VI) with BOC anhydride in presence of a base for example, triethylamine in a suitable organic solvent such as dichloromethane:

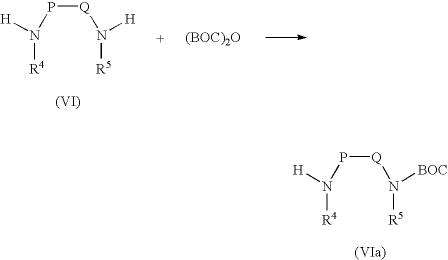

in which R⁴, R⁵, P and Q, are as defined in compounds of formula (II). Certain compounds of formula (VIa) are prepared from compounds of formula (VIb):

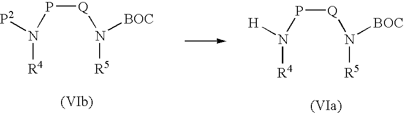

in which P² is a suitable amine protecting group, such as trityl. R⁴, R⁵, P and Q, are as defined in formula (I) or protected derivatives thereof. The trityl protecting group can selectively be removed by reacting with acid such as dilute HCl in a suitable organic solvent such as ethanol.

Compounds of formula (VIb) can be formed as outlined in Scheme 2:

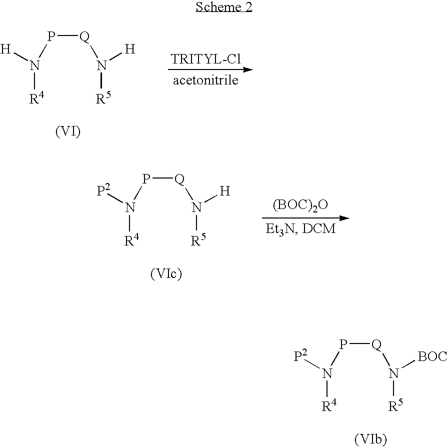

in which R⁴, R⁵, P, Q, and P² are as defined previously for compounds of formula (I) or protected derivatives thereof. P² is defined as for compounds of formula (VIb).

Compounds of formula (V), in which V is CCR¹R² where R¹ and R² are hydrogen can be synthesised as outlined in Scheme 3:

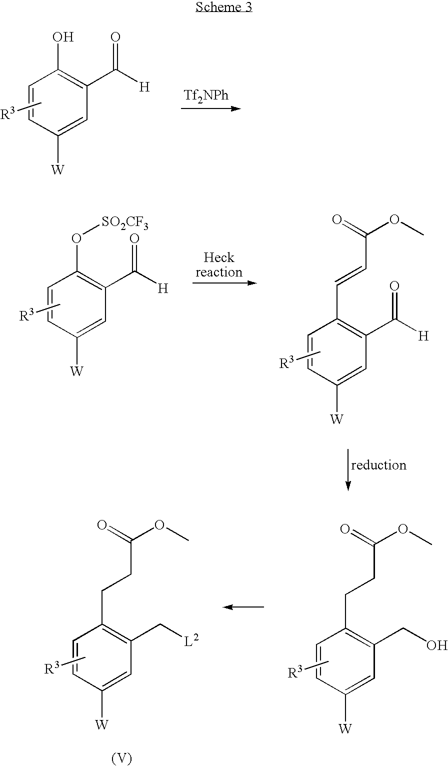

in which R³ and W are as defined for compounds of formula (I) or protected derivatives thereof. L² is defined as for compounds of formula (V).

The hydroxyl group is converted to a leaving group preferably triflate using a suitable reagent, such as phenyl triflamide in the presence of a base such as triethylamine in a suitable organic solvent, suitably DMF. This intermediate then undergoes a Heck reaction with an acrylate, such as methyl acrylate. The alkene moiety and the aldehyde are both reduced using hydrogenation conditions, suitably catalysed by platinum on charcoal. The resulting hydroxy methyl group is converted to a suitable leaving group by reacting with methane sulfonyl chloride in dichloromethane in the presence of a base such as triethylamine. A mixture of both chloro compound and mesylate (V) is obtained. The mixture can be separated or used directly to react with compounds of formula (VI).

Compounds of formula (V) in which V is CH₂COOH can be synthesised as outlined in Scheme 4:

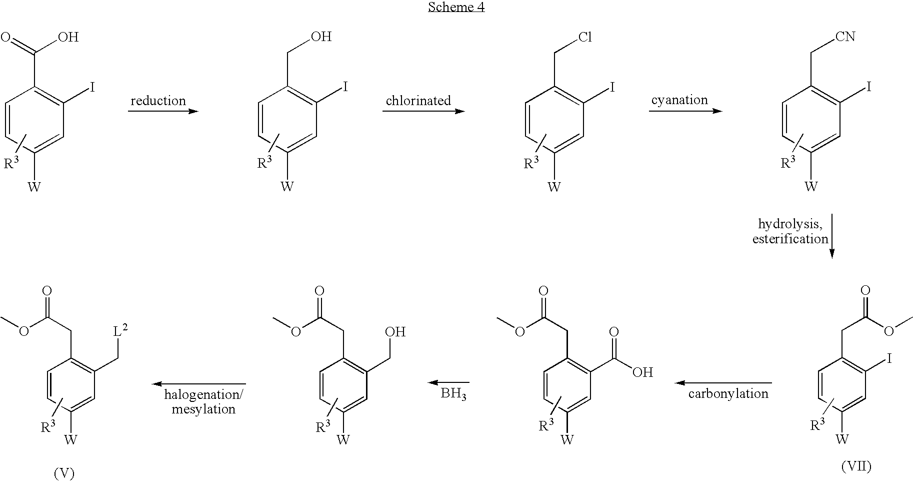

in which W and R³ are as outlined for compounds of formula (I) or protected derivatives thereof. L² is defined as for compounds of formula (V).

The benzoic acid starting material is converted to the alcohol using a reducing agent, preferably, borane in a suitable organic solvent such as THF. The alcohol is then halogenated using a suitable chlorinating agent such as thionyl chloride in the presence of DMF in a solvent such as DCM; subsequent reaction with sodium or potassium cyanide gives the nitrile. The nitrile is then hydrolysed in aqueous potassium hydroxide at elevated temperatured, preferably 100° C. At this stage the acid can be esterified using standard procedures, such as stirring with trimethylsilyl chloride in methanol.

The aryl iodide (VII) can undergo a carbonylation reaction to form the acid by reacting with sodium formate and acetic anhydride and palladium catalysis. Preferred catalyst is bis(dibenzylideneacetone)palladium (0), in a suitable organic solvent such as DMF at elevated temperatures, preferably 80° C. The acid is reduced to the benzyl alcohol using borane as described earlier. The resulting alcohol is activated by mesylation or halogenation using standard procedures known by those skilled in the art. When the compound is mesylated using methane sulfonyl chloride, often a mixture of both mesylate and benzyl chloride is obtained. This mixture can be used directly—as described previously.

Some compounds of formula (V) can be prepared by reacting a compound of formula (Va) with a solution of HBr in an alcoholic solvent such as ethanol at low temperatures, preferably 0° C. in a polar organic solvent, such as ethanol or methanol;

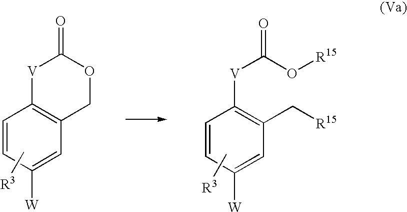

in which V, W, R³ and R¹⁵ are as defined for compounds of formula (II).

Certain compounds of formula (II) can also be prepared as outlined in Scheme 5:

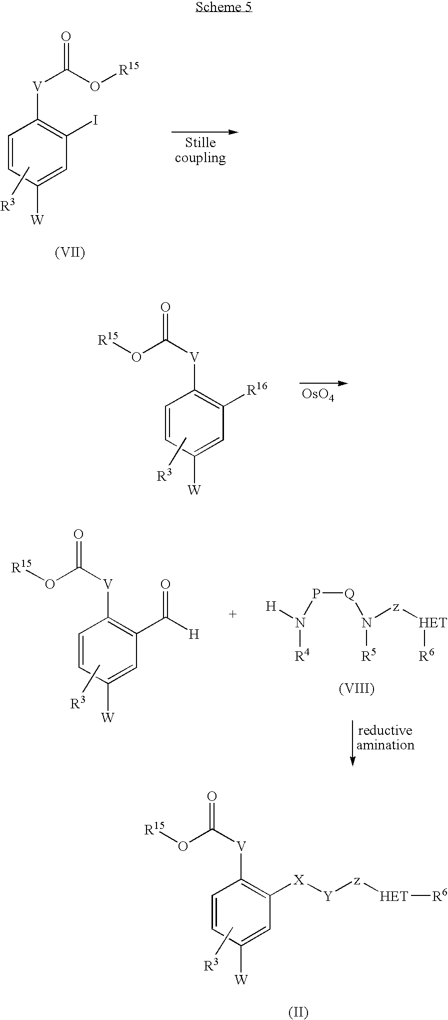

in which R³, V, W and R¹⁵ are a soutlined for compounds of formula (II) or protected derivatives thereof. The aryl iodide (VII) can undergo a Stille coupling reaction with vinyltributyltin in the presence of a suitable palladium catalyst at elevated temperatures, preferably 85-100° C. The alkene is converted to the aldehyde by reaction with osmium tetroxide in suitable solvents such as tertiary butanol, THF and water. The aldehyde can then be reacted with compounds of formula (VIII), under reductive amination conditions. Preferably reacting in the presence of sodiumtriacetoxy borohydride in a suitable organic solvent, such as THF or DCM.

Compounds of formula (VIII) can be prepared from compounds of formula (VI), by reacting the phenolic compound of formula (V) with L²C(R¹, R²)CO₂R¹⁵ in the presence of a base such as potassium carbonate in a suitable solvent such as DMF.

Compounds of formula (VIII) can be prepared from compounds of formula (VI) by reacting with a compound of formula (IV) as described previously in Scheme 1:

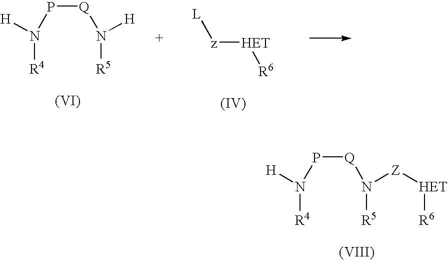

The amino group of compounds of formula (VI) may need to be protected prior to reaction with compounds of formula (IV). Suitable protecting groups are BOC, trityl or benzyl, which can be removed readily using the procedures described previously. Some protected compounds of formula (VI) are commercially available.
Compounds of formula (IX) can be prepared from compounds of formula (I) by coupling with a compound of formula (X) as shown in Scheme 6:

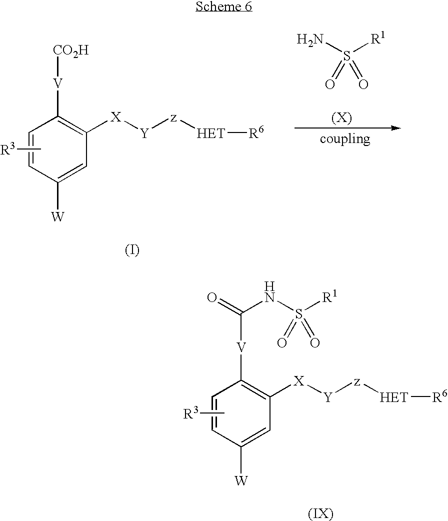

in which R¹, R³, R⁶, V, W, X, Y, Z and HET are as defined in compounds of formula (I) or protected derivatives thereof. The coupling can be carried out using standard coupling methods. For example, compounds of formula (I) can be converted to the acid chloride using a reagent such as oxalyl chloride and subsequently reacted with an acyl sulfonamide of formula (X) using a suitable base such as hunigs base in a suitable solvent such as DCM. Alternatively compounds of formula (I) can be directly coupled with acyl sulfonamides of formula (X) using a suitable coupling agent such as PyBOP or HATU or CDI with a suitable base such as Hunigs base or DBU in a suitable solvent such as DCM or THF. In a further aspect, the present invention provides the use of a compound of formula (I), a prodrug, pharmaceutically acceptable salt or solvate thereof for use in therapy.

The compounds of formula (I) have activity as pharmaceuticals, in particular as modulators of CRTh2 receptor activity, and may be used in the treatment (therapeutic or prophylactic) of conditions/diseases in human and non-human animals which are exacerbated or caused by excessive or unregulated production of PGD₂ and its metabolites. Examples of such conditions/diseases include

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; esophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Thus, the present invention provides a compound of formula (I), or a pharmaceutically-acceptable salt or solvate thereof, as hereinbefore defined for use in therapy.

Preferably the compounds of the invention are used to treat diseases in which the chemokine receptor belongs to the CRTh2 receptor subfamily.

Particular conditions which can be treated with the compounds of the invention are asthma, rhinitis and other diseases in which raised levels of PGD₂ or its metabolites. It is preferred that the compounds of the invention are used to treat asthma.

In a further aspect, the present invention provides the use of a compound of formula (I), or a pharmaceutically acceptable salt or solvate thereof, as hereinbefore defined in the manufacture of a medicament for use in therapy.

In a further aspect, the present invention provides the use of a compound or formula (I), or a pharmaceutically acceptable salt or solvate thereof, as hereinbefore defined in the manufacture of a medicament for use in therapy in combination with drugs used to treat asthma and rhinitis (such as inhaled and oral steroids, inhaled β2-receptor agonists and oral leukotriene receptor antagonists).

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases rheumatoid arthritis, psoriasis, inflammatory bowel disease, COPD, asthma and allergic rhinitis the compounds of the invention may be combined with agents such as tumour necrosis factor alpha (TNF-α) inhibitors such as anti-TNF monoclonal antibodies (for example Remicade, CDP-870 and adalimumab) and TNF receptor immunoglobulin molecules (such as Enbrel); non-selective cyclo-oxygenase (COX)-1/COX-2 inhibitors whether applied topically or systemically (such as piroxicam, diclofenac, propionic acids such as naproxen, flubiprofen, fenoprofen, ketoprofen and ibuprofen, fenamates such as mefenamic acid, indomethacin, sulindac, azapropazone, pyrazolones such as phenylbutazone, salicylates such as aspirin), COX-2 inhibitors (such as meloxicam, celecoxib, rofecoxib, valdecoxib, lumarocoxib, parecoxib and etoricoxib); glucocorticosteroids (whether administered by topical, oral, intramuscular, intravenous, or intra-articular routes); methotrexate, lefunomide; hydroxychloroquine, d-penicillamine, auranofin or other parenteral or oral gold preparations.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonist for leukotrienes (LT)B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a phosphodiesterase (PDE) inhibitor such as the methylxanthanines including theophylline and aminophylline; and selective PDE isoenzyme inhibitors including PDE4 inhibitors and inhibitors of the isoform PDE4D, and inhibitors of PDE5.
The present invention still further relates to the combination of a compound of the invention together with histamine type 1 receptor antagonists such as cetirizine, loratadine, desloratadine, fexofenadine, acrivastine, terfenadine, astemizole, azelastine, levocabastine, chlorpheniramine, promethazine, cyclizine, and mizolastine applied orally, topically or parenterally.
The present invention still further relates to the combination of a compound of the invention together with a gastroprotective histamine type 2 receptor antagonist.
The present invention still further relates to the combination of a compound of the invention with antagonists of the histamine type 4 receptor.
The present invention still further relates to the combination of a compound of the invention together with an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride, and ethylnorepinephrine hydrochloride.
The present invention still further relates to the combination of a compound of the invention together with anticholinergic agents including muscarinic receptor (M1, M2, and M3) antagonists such as atropine, hyoscine, glycpyrrrolate, ipratropium bromide; tiotropium bromide; oxitropium bromide; pirenzepine; and telenzepine.
The present invention still further relates to the combination of a compound of the invention together with a beta-adrenoceptor agonist (including beta receptor subtypes 1-4) such as isoprenaline, salbutamol, formoterol, salmeterol, terbutaline, orciprenaline, bitolterol mesylate, and pirbuterol.
The present invention still further relates to the combination of a compound of the invention together with a chromone, including sodium cromoglycate and nedocromil sodium.
The present invention still further relates to the combination of a compound of the invention together with an insulin-like growth factor type I (IGF-1) mimetic.
The present invention still further relates to the combination of a compound of the invention together with an inhaled glucocorticoid, such as flunisolide, triamcinolone acetonide, beclomethasone dipropionate, budesonide, fluticasone propionate, ciclesonide, and mometasone furoate.
The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12.
The present invention still further relates to the combination of a compound of the invention together with modulators of chemokine receptor function such as antagonists of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.
The present invention still further relates to the combination of a compound of the invention together with a cytokine or modulator of cytokine function, including alpha-, beta-, and gamma-interferon; interleukins (IL) including IL1 to 15, and interleukin antagonists or inhibitors, including agents which act on cytokine signalling pathways.
The present invention still further relates to the combination of a compound of the invention together with an immunoglobulin (Ig) or Ig preparation or an antagonist or antibody modulating Ig function such as anti-IgE (omalizumab).
The present invention still further relates to the combination of a compound of the invention together with other systemic or topically-applied anti-inflammatory agents including thalidomide and derivatives, retinoids, dithranol, and calcipotriol.
The present invention still further relates to the combination of a compound of the invention together with an antibacterial agent including penicillin derivatives, tetracyclines, macrolides, beta-lactams, fluoroquinolones, and inhaled aminoglycosides; and antiviral agents including acyclovir, famciclovir, valaciclovir, ganciclovir, cidofovir; amantadine, rimantadine; ribavirin; zanamavir and oseltamavir; protease inhibitors such as indinavir, nelfinavir, ritonavir, and saquinavir; nucleoside reverse transcriptase inhibitors such as didanosine, lamivudine, stavudine, zalcitabine, zidovudine; non-nucleoside reverse transcriptase inhibitors such as nevirapine, efavirenz.
The present invention still further relates to the combination of a compound of the invention together with cardiovascular agents such as calcium channel blockers, beta-adrenoceptor blockers, angiotensin-converting enzyme (ACE) inhibitors, angiotensin-2 receptor antagonists; lipid lowering agents such as statins, and fibrates; modulators of blood cell morphology such as pentoxyfylline; thrombolytics, and anticoagulants including platelet aggregation inhibitors.
The present invention still further relates to the combination of a compound of the invention together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase), and anti-Alzheimer's drugs such as donepezil, tacrine, COX-2 inhibitors, propentofylline or metrifonate.
The present invention still further relates to the combination of a compound of the invention together with agents for the treatment of acute and chronic pain, including centrally and peripherally-acting analgesics such as opioid analogues and derivatives, carbamazepine, phenyloin, sodium valproate, amitryptiline and other antidepressant agents, and non-steroidal anti-inflammatory agents.
The present invention still further relates to the combination of a compound of the invention together with parenterally or topically-applied local anaesthetic agents such as lignocaine.

The present invention still further relates to the combination of a compound of the invention together with (i) tryptase inhibitors; (ii) platelet activating factor (PAF) antagonists; (iii) interleukin converting enzyme (ICE) inhibitors; (iv) IMPDH inhibitors; (v) adhesion molecule inhibitors including VLA-4 antagonists; (vi) cathepsins; (vii) MAP kinase inhibitors; (viii) glucose-6 phosphate dehydrogenase inhibitors; (ix) kinin-B.sub1.- and B.sub2.-receptor antagonists; (x) anti-gout agents, e.g., colchicine; (xi) xanthine oxidase inhibitors, e.g., allopurinol; (xii) uricosuric agents, e.g., probenecid, sulfinpyrazone, and benzbromarone; (xiii) growth hormone secretagogues; (xiv) transforming growth factor (TGFβ); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) Tachykinin NK.sub1. and NK.sub3. receptor antagonists selected from the group consisting of NKP-608C; SB-233412 (talnetant); and D-4418; (xx) elastase inhibitors selected from the group consisting of UT-77 and ZD-0892; (xxi) TNF□ converting enzyme inhibitors (TACE); (xxii) induced nitric oxide synthase inhibitors (iNOS) or (xxiii) chemoattractant receptor-homologous molecule expressed on TH2 cells, (CRTH2 antagonists) (xxiv) inhibitors of P38

The compounds of the present invention may also be used in combination with anti-osteoporosis agents including hormonal agents such as raloxifene, and biphosphonates such as alendronate.
The compounds of the invention may also be used in combination with existing therapeutic agents for the treatment of osteoarthritis. Suitable agents to be used in combination include standard non-steroidal anti-inflammatory agents (hereinafter NSAIDs) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics, and intra-articular therapies such as corticosteroids and hyaluronic acid derivatives, and nutritional supplements such as glucosamine.
The compounds of the invention can also be used in combination with existing therapeutic agents for the treatment of cancer. Suitable agents to be used in combination include:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), estrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab, compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.
In a still further aspect, the present invention provides the use of a compound of formula (I), or a pharmaceutically acceptable salt or solvate thereof, as hereinbefore defined in the manufacture of a medicament for the treatment of human diseases or conditions in which modulation of CRTh2 receptor activity is beneficial.

In the context of the present specification, the term “therapy” also includes “prophylaxis” unless there are specific indications to the contrary. The terms “therapeutic” and “therapeutically” should be construed accordingly.

The invention still further provides a method of treating diseases mediated by PGD2 or its metabolites wherein the prostanoid binds to its receptor (especially CRTh2) receptor, which comprises administering to a patient a therapeutically effective amount of a compound of formula (I), or a pharmaceutically acceptable salt, solvate or prodrug thereof, as hereinbefore defined.

The invention also provides a method of treating an inflammatory disease, especially psoriasis, in a patient suffering from, or at risk of, said disease, which comprises administering to the patient a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

For the above-mentioned therapeutic uses the dosage administered will, of course, vary with the compound employed, the mode of administration, the treatment desired and the disorder indicated.

The compound of formula (I), prodrugs and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as herein before defined, in association with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The invention will now be illustrated by the following non-limiting examples in which, unless stated otherwise:

(i) when given, ¹H NMR data is quoted in the form of delta values for major diagnostic protons, given in parts per million (ppm) relative to tetramethylsilane (TMS) as an internal standard;
(ii) mass spectra (MS): generally only ions which indicate the parent mass are reported, and unless otherwise stated the mass ion quoted is the positive mass ion—(M+H)⁺;
(iii) the title compounds of the examples and methods were named using the ACD/name and ACD/name batch (version 6.0) from Advanced Chemical Development Inc, Canada;
(iv) unless stated otherwise, reverse phase HPLC was conducted using a Symmetry, NovaPak or Ex-Terra reverse phase silica column;
(v) solvents were dried with MgSO₄ or Na₂SO₄
(vi) the following abbreviations are used:
aq aqueous
DCM dichloromethane
DMF N,N-dimethylformamide
ether diethyl ether
EtOAc ethyl acetate
EtOH ethanol
h hour
HATU O-(7-Azabenzotriazol-1-yl)-N,N,N′,N′-tetramethyluronium hexafluorophosphonate
HCl hydrochloric acid
SCX sulphonic acid resin
NaOH sodium hydroxide
K₂CO₃ potassium carbonate
KOH potassium hydroxide
MeOH methanol
NaHCO₃ sodium hydrogen carbonate
NMP N-methylpyrrolidine
Pd(dppf)Cl_{2 [}1,1′-Bis(diphenylphosphino)ferrocene]dichloropalladium(II), complex with dichloromethane
Pd₂ dba₃ bis(dibenzylideneacetone)palladium (0)
RPHPLC reverse phase high performance liquid chromatography
RT room temperature
TFA trifluoroacetic acid
THF tetrahydrofuran

### EXAMPLE 1

### Sodium 3-(2-{[4-(benzylsulfonyl)piperazin-1-yl]methyl}-4-chlorophenyl)propanoate

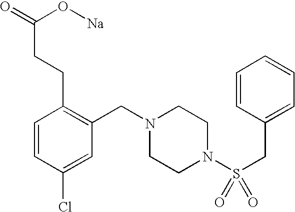

### (i) 4-chloro-2-formylphenyl trifluoromethanesulfonate

Phenyl triflimate (Tf₂NPh) (3.05 g) was added portionwise to a solution of 5-chloro-2-hydroxybenzaldehyde (1.13 g) and triethylamine (1.2 ml) in DMF (5 ml) and stirred for 4 h. The reaction was quenched with water and then extracted with ether. The ether layer was washed with water, brine, then dried (MgSO₄) and evaporated under reduced pressure. The residue was purified by chromatography on silica (eluent 4:1 then 2:1 petrol/DCM) to give the sub-title compound, yield 1.89 g

¹H NMR CDCl₃: δ 10.23 (1H, s), 7.97 (1H, d), 7.67 (1H, dd), 7.36 (1H, d).

### (ii) methyl (2E)-3-(4-chloro-2-formylphenyl)acrylate

A mixture of methyl acrylate (1 ml), the product of step (i) (1.36 g), triethylamine (1.3 ml) and Pd(dppf)Cl₂ (35 mg) in THF (4 ml) was heated at reflux for 8 h. Water was added and extracted with ether. The ether layer was washed with water, brine, then dried (MgSO₄) and evaporated under reduced pressure. The residue was purified by chromatography on silica (eluent 2:1 petrol/ether) to give the sub-title compound, yield 410 mg

¹H NMR CDCl₃: δ 10.23(1H, s), 8.44 (1H, d), 7.86 (1H,d), 7.59 (2H, d), 6.38 (1H, d), 3.84 (3H, s).

### (iii) methyl 3-[4-chloro-2-(hydroxymethyl)phenyl]propanoate

A mixture of the product of step (ii) (390 mg), 5% Platinum on carbon (151 mg) in EtOAc (10 ml) was stirred under 4 ATM of hydrogen for 2 days. The reaction was filtered and the filtrate was evaporated under reduced pressure to give the sub-title compound as a yellow oil (376 mg).

¹H NMR CDCl₃: δ 7.39 (1H, d), 7.22 (1H, dd), 7.12 (1H,d ), 4.70 (2H, s), 4.63 (3H, s), 2.97 (2H, t), 2.66 (2H, t).

### (iv) methyl 3-[4-chloro-2-(chloromethyl)phenyl]propanoate

Methane sulfonyl chloride (0.18 ml) was added to a solution of the product of step (iii) (437 mg) and triethylamine (0.4 ml) in DCM (4 ml) and stirred for 3 h. Water was added and the mixture was extracted with DCM. The organic phase was dried (MgSO₄) and evaporated under reduced pressure. The residue was purified by chromatography on silica (eluent 1:2 petrol/ether) to give the sub-title compound, yield 273 mg.

¹H NMR CDCl₃: δ 7.34 (1H, d), 7.24 (1H, dd), 7.16 (1H,d), 4.60 (2H, s), 3.69 (3H, s), 3.03 (2H, t), 2.66 (2H, t).

### (iva) (methyl 3-(4-chloro-2-{[(methylsulfonyl)oxy]methyl}phenyl)propanoate)

The mesylate was also obtained, yield 170 mg.

¹H NMR CDCl₃: δ 7.39(1H, d), 7.33 (1H, dd), 7.20 (1H, d), 5.28 (2H, d), 3.67 (3H, s), 3.01 (3H, s), 3.0 (2H, t), 2.64 (2H, t).

¹H NMR CDCl₃: δ 7.41-6.91 (8H, m), 4.21 (3H, t), 4.14 (2H, s), 3.66 (2H, s), 3.12 (4H, t), 2.93 (2H, t), 2.58 (2H, t), 2.40 (4H, t), 1.26 (3H, t).

### (v) tert-butyl 4-(1-benzylsulfonyl)piperazine-1-carboxylate

Triethylamine (6 ml) was added to a stirred solution of tert-butyl piperazine-1-carboxylate (7.75 g) and benzylsulfonyl chloride (7.92 g) in DCM, and then stirred overnight. The solvent was evaporated under reduced pressure and the residue was dissolved in EtOAc, washed with water, dried (MgSO₄) and evaporated under reduced pressure to give the sub-title compound as a white solid, yield 15.36 g

MS: ESI(−ve) 339 (M−H)

### (vi) 1-(benzylsulfonyl)piperazine

TFA (10 ml) was added to a solution of the product of step (v) (15.36 g) in DCM (20 ml) and stirred overnight. The reaction mixture was concentrated under reduced pressure to give an oil, which was then triturated with diethyl ether to give a pink solid, yield 5.61 g.

¹H NMR DMSO-D6: δ 8.74 (1H, s, br), 7.46-7.39 (5H, m), 4.55 (2H, s), 3.29 (4H, t), 3.09 (4H, t).

### (vii) methyl 3-(2-{[4-(benzylsulfonyl)piperazin-1-yl]methyl}-4-chlorophenyl)propanoate

A mixture of the product of step (iv) (35 mg), the product of step (iva) (170 mg), the product of step (vi) (293 mg) and K₂CO₃ (241 mg) in ethanol (4 ml) was stirred for 2.5 days. Aqueous ammonium chloride was added and the reaction was extracted with DCM, dried (MgSO₄) and evaporated under reduced pressure. The residue was purified by chromatography on silica (eluent 2:3 petrol/ether) to give the sub-title compound was obtained as a mixture of methyl and ethyl esters, yield 206 mg.

### (viii) Sodium 3-(2-{[4-(benzylsulfonyl)piperazin-1-yl]methyl}-4-chlorophenyl)propanoate

A solution of the product of step (vii) (204 mg), NaOH (0.44 ml), THF (2 ml), methanol (2 ml) was stirred for 3 h. The solvent was removed under reduced pressure, the residue was washed with ether and then recrystallised from MeCN/MeOH to give the title compound as a white solid, yield 185 mg.

¹H NMR DMSO-D6: δ 7.42-7.14 (8H, m), 4.41 (2H, s), 3.49 (2H, s), 3.14 (4H, s), 2.78 (2H, t), 2.41 (4H, s), 2.08 (2H, t).

MS: ESI(+ve) 439 (M+1)

### EXAMPLE 2

### 3-(2-{[(3S)-4-(benzylsulfonyl)-3-methylpiperazin-1-yl]methyl}-4-chlorophenyl)propanoic acid

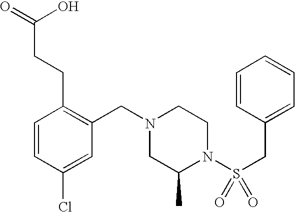

### (i) tert-butyl (3S)-3-methylpiperazine-1-carboxylate

Triethylamine (2.85 ml) was added to a solution of (S)-2-methyl piperazine (1 g) in methanol (25 ml), this was followed by portionwise addition of BOC anhydride (2.18 g). The reaction mixture was stirred for 17 h, then concentrated under reduced pressure. Water was added to the residue and extracted EtOAc (×3), dried (MgSO₄) and evaporated under reduced pressure. The residue was purified by chromatography on silica (eluent EtOAc, then 9:1:1 EtOAc:MeOH:NH₃) to give the sub-title compound as a colourless oil, yield 1.3 g.

¹H NMR CDCl₃: δ 4.04-3.82 (2H, m), 2.95 (1H, d), 2.81-2.66 (3H, m), 2.48-2.32 (1H, m), 1.47 (9H, s), 1.05 (3H, d).

### (ii) tert-butyl (3S)-4-(benzylsulfonyl)-3-methylpiperazine-1-carboxylate

A mixture of the product of step (i) (650 mg), K₂CO₃ (1.15 g), DCM (6 ml) and water (6 ml) were stirred vigorously. Benzylsulfonyl chloride (992 mg) was added portionwise over 2 min and then stirred for 4.5 h. The reaction was diluted with DCM, washed with water, brine, dried (MgSO₄) and evaporated under reduced pressure to give the sub-title compound as a white solid, yield 1.06 g.

¹H NMR CDCl₃: δ 7.38 (5H, s), 4.20 (2H, d), 4.04-3.82 (2H, m), 2.95 (1H, d), 2.81-2.66 (3H, m), 2.48-2.32 (1H, m), 1.47 (9H, s), 1.05 (3H, d).

### (iii) (2S)-1-(benzylsulfonyl)-2-methylpiperazine, trifluoroacetic acid salt

The sub-title compound was prepared by the method of example 1 step (vi) using the product of step (ii) to give an off-white solid, yield 1.03 g.

¹H NMR CDCl₃: δ 7.41 (5H, s), 4.24 (2H, d), 4.11-3.98 (1H, m), 3.4-3.26 (2H, m), 3.11 (1H, d), 2.97 (2H, s), 2.81-2.65 (1H, m), 1.32 (3H, d).

### (iv) methyl 3-(4-chloro-2-{[(methylsulfonyl)oxy]methyl}phenyl)propanoate

Methanesulfonyl chloride (0.39 ml) was added to a solution of the product of example 1 step (iii) (946 mg) and triethyl amine (0.85 ml) in DCM (10 ml), and then stirred for 3 h. Water was added and the mixture was extracted with DCM (×3). The combined organic extracts were dried (MgSO₄) and evaporated under reduced pressure to give a 2.5:1 mixture of chloride and mesylate as for example 1 step (iv) and (iva). The mixture was used directly without purification.

### (v) methyl 3-(2-{[(3S)-4-(benzylsulfonyl)-3-methylpiperazin-1-yl]methyl}-4-chlorophenyl)propanoate

The mixture of products from step (iv) (200 mg), the product of step (iii) (332 mg) and K₂CO₃ (263 mg) in DMF (5 ml) were charged to a flask and stirred for 2.5 days. The reaction was diluted with water, extracted with EtOAc (×3). The combined organic extracts were dried (MgSO₄) and evaporated under reduced pressure. The residue was purified by chromatography on silica (eluent 3:2 then 2:3 iso-hexane:ether) to give the sub-title compound as a colourless oil, yield 211 mg.

¹H NMR CDCl₃: δ 7.39 (5H, s), 7.28-7.14 (2H, m), 7.09 (1H, d), 4.19 (2H, d), 3.92-3.81 (1H, m), 3.67 (3H, m), 3.43-3.3 (2H, m), 3.27-3.18 (1H, m), 3.09 (1H, td), 2.97 (2H, t), 2.65-2.55 (3H, m), 2.52 (1H, d), 2.14 (1H, dd), 1.93 (1H, td), 1.24 (3H, d).

### (vi) 3-(2-{[(3S)-4-(benzylsulfonyl)-3-methylpiperazin-1-yl]methyl}-4-chlorophenyl)propanoic acid

The title compound was prepared by the method of example 1 step (viii). The product was isolated by reverse phase HPLC.

¹H NMR DMSO-D6: δ 7.45-7.33 (5H, m), 7.3 (1H, s), 7.23 (2H, s), 4.46-4.33 (2H, m), 3.84-3.71 (1H, m), 3.48-3.25 (3H, m), 3.06 (1H, t), 2.86 (2H, t), 2.63-2.39 (4H, m), 2.21 (1H, dd), 1.99-1.86 (1H, m), 1.17 (3H, d).

MS: APCI(+ve) 451 (M+H)

### EXAMPLE 3

### Sodium 3-(4-chloro-2-{[(3S)-3-methyl-4-(phenylsulfonyl)piperazin-1-yl]methyl}phenyl)propanoate

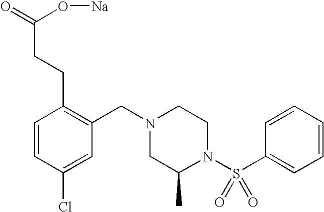

### (i) tert-butyl (3S)-3-methyl-4-(phenylsulfonyl)piperazine-1-carboxylate

The product of example 2 step (i) (0.65 g) was dissolved in DCM and triethylamine (1.36 ml) was added, followed by dropwise addition of benzenesulfonyl chloride (0.5 ml), and then stirred for 24 h. Further benzene sulfonyl chloride (0.15 ml) was added and stirred for 2 h. The reaction mixture was concentrated under reduced pressure. The residue was purified by chromatography on silica (eluent 8:2 iso-hexane: EtOAc) to give the sub-title compound as a pale yellow solid, yield 1 g.

¹H NMR CDCl₃: δ 7.81 (2H, dt), 7.62-7.47 (3H, m), 4.17-4.07 (2H, m), 3.86-3.71 (1H, m), 3.62 (1H, d), 3.12 (1H, dt), 3.04-2.88 (1H, m), 2.87-2.7 (1H, m), 1.42 (9H, s), 1.01 (3H, d).

### (ii) (2S)-2-methyl-1-(phenylsulfonyl)piperazine

The sub-title compound was prepared by the method of example 1 step (vi) using the product of step (i).

¹H NMR CDCl₃: δ 7.81 (2H, d), 7.64 (1H, t), 7.56 (2H, t), 4.41-4.28 (1H, m), 3.86 (1H, d), 3.58-3.4 (1H, m), 3.33 (1H, d), 3.14 (2H, s), 3.06-2.9 (1H, m), 1.23 (3H, d).

### (iii) methyl 3-(4-chloro-2-{[(3S)-3-methyl-4-(phenylsulfonyl)piperazin-1-yl]methyl}phenyl)propanoate

The sub-title compound was prepared by the method of example 2 step (v) using the product of example 2 step (iv) and the product of step (ii).

¹H NMR CDCl₃: δ 7.82 (2H, d), 7.62-7.45 (3H, m), 7.23-7.05 (3H, m), 4.17-4.05 (1H, m), 3.65 (3H, s), 3.62-3.58 (1H, m), 3.44-3.28 (2H, m), 3.19 (1H, td), 2.96 (2H, t), 2.68 (1H, d), 2.63-2.48 (3H, m), 2.21 (1H, dd), 2.03 (1H, td), 1.12 (3H, d).

### (iv) Sodium 3-(4-chloro-2-{[(3S)-3-methyl-4-(phenylsulfonyl)piperazin-1-yl]methyl}phenyl)propanoate

The title compound was prepared by the method of example 1 step (viii). The product was isolated by reverse phase HPLC.

¹H NMR DMSO-D6: δ 7.8 (2H, d), 7.68 (1H, t), 7.61 (2H, t), 7.24 (1H, s), 7.18 (2H, s), 4.03-3.94 (1H, m), 3.57 (1H, d), 3.35 (2H, s), 3.11 (2H, t), 2.75 (2H, t), 2.64 (1H, d), 2.15 (2H, t), 2.0 (1H, dd), 1.89 (1H, td), 1.03 (3H, d).

MS: APCI(+ve) 437 (M+H)

### EXAMPLE 4

### 3-(4-chloro-2-{[(3S)-3-methyl-4-(phenylacetyl)piperazin-1-yl]methyl}phenyl)propanoic acid

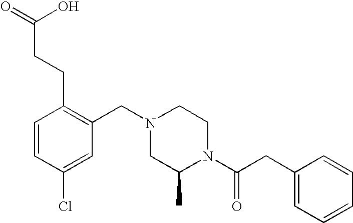

### (i) tert-butyl (3S)-3-methyl-4-(phenylacetyl)piperazine-1-carboxylate

The sub-title compound was prepared by the method of example 2 step (ii) using the product of example 2 step (i), phenylacetyl chloride and NaHCO₃ as base instead of K₂CO₃.

¹H NMR CDCl₃: δ 7.32 (2H, t), 7.28-7.18 (3H, m), 4.87-4.76 (1H, m), 4.49-4.38 (1H, m), 4.18-3.92 (1H, m), 3.87-3.68 (2H, m), 3.01-2.81 (2H, m), 2.8-2.68 (1H, m), 2.62-2.5 (1H, m), 1.45 (9H, s), 1.18-1.05 (3H, m).

### (ii) (2S)-2-methyl-1-phenylacetyl)piperazine

The sub-title compound was prepared by the method of example 1 step (vi) using the product of step (i).

¹H NMR CDCl₃: δ 7.32 (2H, t), 7.27-7.18 (3H, m), 4.84-4.64 (1H, m), 4.55-4.32 (1H, d), 4.06-3.87 (1H, m), 3.75 (2H, s), 3.27-3.13 (2H, m), 3.05 (1H, dd), 2.83 (1H, td), 1.15 (3H, d).

### (iii) methyl 3-(4-chloro-2-{[(3S)-3-methyl-4-(phenylacetyl)piperazin-1-yl]methyl}phenyl)propanoate

The sub-title compound was prepared by the method of example 1 step (vi) using the product of step (i).

¹H NMR CDCl₃: δ 7.32 (2H, t), 7.27-7.18 (4H, m), 7.17 (1H, d), 7.1 (1H, d), 4.83-4.74 (1H, m), 4.44 (1H, d), 4.06-3.98 (1H, m), 3.71 (2H, s), 3.66 (3H, s), 3.57-3.5 (1H, m), 3.42-3.16 (2H, m), 2.98 (2H, t), 2.95-2.86 (1H, m), 2.81-2.73 (1H, m), 2.68-2.53 (3H, m), 2.2-2.13 (1H, m), 2.05-1.92 (1H, m), 1.85-1.76 (1H, m).

MS: ESI(+ve) 429 (M+H)

### (iv) 3-(4-chloro-2-{[(3S)-3-methyl-4-(phenylacetyl)piperazin-1-yl]methyl}phenyl)propanoic acid

The title compound was prepared by the method of example 1 step (viii) using the product of step (iii).

¹H NMR CDCl₃: δ 7.32-7.28 (3H, m), 7.25-7.16 (5H, m), 4.59-4.50 (1H, m), 4.23-4.15 (1H, m), 3.77-3.62 (2H, m), 3.41 (2H, q), 3.11 (1H, t), 2.88 (2H, t), 2.8-2.55 (2H, m), 2.48 (2H, t), 2.05 (1H, dd), 1.85 (1H, dd), 1.13-1.05 (3H, m).

MS: ESI(+ve) 415 (M+H)

### EXAMPLE 5

### 3-[4-chloro-2-({(3S)-3-methyl-4-[(4-methylbenzyl)sulfonyl]piperazin-1-yl}methyl)phenyl]propanoic acid

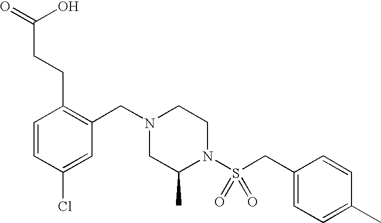

### (i) (3S)-3-methyl-1-(triphenylmethyl)-piperazine

(S)+-2-methylpiperazine (10 g) was dissolved in acetonitrile (140 ml) and cooled to 5-10° C. whereupon triethylamine (35 ml) was added, followed by drop wise addition of a solution of trityl chloride (27.9 g) in DCM (80 ml). The reaction was stirred for 1 h at RT. The resulting slurry was cooled to approximately 0° C. then filtered. The filtrate was evaporated in vacuo and the residue was purified by chromatography (silica, 0-1% MeOH/DCM as eluent), then triturated with ether to give the sub-title compound as a white solid yield, 29.8 g.

¹H NMR CDCl₃: δ 7.49-7.37 (6H, m), 7.26 (6H, t), 7.15 (3H, t), 3.38-3.28 (1H, m), 3.22 (1H, dd), 3.11-2.99 (3H, m), 1.74-1.6 (1H, m), 1.44-1.3 (1H, m), 1.11 (3H, d).

### (ii) (2S)-1-piperazinecarboxylic acid, 2-methyl-4-(triphenylmethyl)-1,1-dimethylethyl ester Triethylamine (24.3 ml) was added to a solution of the product from part a) (29.8 g) in methanol (350 ml). BOC-anhydride (19 g) was then added to the reaction mixture and stirred overnight. The solvents were evaporated under reduced pressure. The residue was partitioned between EtOAc and saturated brine. The organic layer was separated and washed with brine, dried (Na₂SO₄) the concentrated in vacuo to give the sub-title compound as a foam, yield, 35 g.

¹H NMR (CDCl₃) δ 7.49-7.16 (15H, m), 4.13 (1H, t), 3.74 (1H, d), 3.33 (1H, t), 2.97 (4H, m), 1.68 (3H, dd) and 1.33 (9H, s).

### (iii) (2S)-2-methyl-1-piperazinecarboxylic acid-1,1-dimethylethyl ester

2M HCl (50 ml) was added drop wise to a solution of the product of part b) (34 g) in ethanol (1500 ml), the reaction was stirred for 1.5 h. Solid NaHCO₃ (8.4 g) was added and stirred for 1 h, then concentrated under reduced pressure. The residue was purified by chromatography (silica, 5-10% MeOH/DCM as eluent) to remove the by-products, then eluted with 10% MeOH/DCM to give the sub-title compound, yield 16.5 g.

¹H NMR (CDCl₃) δ 4.51 (1H, t), 4.07 (1H, d), 3.46-3.33 (2H, m), 3.21 (1H, d), 3.09 (1H, dd), 2.88 (1H, td) and 1.49-1.43 (12H, m).

### (iv) tert-butyl (2S)-4-[5-chloro-2-(3-methoxy-3-oxopropyl)benzyl]-2-methylpiperazine-1-carboxylate

The sub-title compound was prepared by the method of example 2 step (v) using the product of example 2 step (iv) and the product of step (iii).

¹H NMR (CDCl₃) δ 7.28-7.26 (1H, m), 7.19 (1H, dd), 7.11 (1H, d), 4.25-4.16 (1H, m), 3.80 (1H, d), 3.67 (3H, s), 3.4 (2H, q), 3.09-2.95 (3H, m), 2.7 (1H, d), 2.67-2.57 (3H, m), 2.2 (1H, dd), 1.9 (1H, td), 1.46 (9H, s), 1.20 (3H, d).

### (v) methyl 3-(4-chloro-2-{[(3S)-3-methylpiperazin-1-yl]methyl}phenyl)propanoate, TFA salt

The sub-title compound was prepared by the method of example 1 step (vi) using the product of step (iv).

¹H NMR (CDCl₃) δ 7.44-7.4 (2H, m), 7.24 (1H, s), 4.5 (1H, d), 4.45 (1H, d), 4.01-3.91 (1H, m), 3.76-3.52 (6H, m), 3.62 (3H, s), 2.95 (2H, t), 2.78 (2H, t), 1.48 (13H, d).

### (vi) methyl 3-[4-chloro-2-({(3S)-3-methyl-4-[(4-methylbenzyl)sulfonyl]piperazin-1-yl}methyl)phenyl]propanoate

The product of step (v) (360 mg) was dissolved in DCM (5 ml), this was followed by addition of a solution of NaHCO₃ (218 mg) in water (5 ml). (4-methylphenyl)methane sulfonyl chloride (280 mg) was added portionwise and stirred for 1 day, then additional NaHCO₃ and sulfonyl chloride were added and stirred for 3 days overall. The reaction was diluted with water and extracted with DCM (×3). The combined organic layers were washed (brine), dried (MgSO₄) then concentrated under reduced pressure to give the sub-title compound as a pale yellow oil, yield 210 mg.

MS: ESI(+ve) 479 (M+H)

### (vii) 3-[4-chloro-2-({(3S)-3-methyl-4-[(4-methylbenzyl)sulfonyl]piperazin-1-yl}methyl)phenyl]propanoic acid

The title compound was prepared by the method of example 1 step (viii) using the product of step (vi).

¹H NMR CD₃OD: δ 7.31 (2H, d), 7.25 (1H, s), 7.22-7.15 (4H, m), 4.26 (2H, s), 3.77-3.62 (2H, m), 3.41 (2H, q), 3.11 (1H, t), 2.88 (2H, t), 2.8-2.55 (2H, m), 2.48 (2H, t), 2.34 (3H, s), 2.05 (1H, dd), 1.85 (1H, dd), 1.13-1.05 (3H, m).

MS: ESI(+ve) 415 (M+H)

### EXAMPLE 6

### 3-[4-chloro-2-({(3S)-3-methyl-4-[(3-methylbenzyl)sulfonyl]piperazin-1-yl}methyl)phenyl]propanoic acid

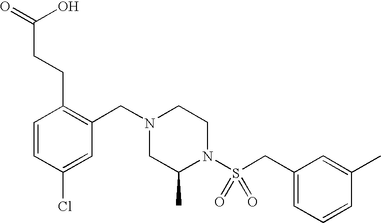

### (i) methyl 3-[4-chloro-2-({(3S)-3-methyl-4-[(3-methylbenzyl)sulfonyl]piperazin-1-yl}methyl)phenyl]propanoate

The product of example 5 step (v) (400 mg) was dissolved in DCM (5 ml), this was followed by addition of a solution of K₂CO₃ (520 mg) in water (4 ml). (3-methylphenyl)methane sulfonyl chloride (307 mg) was added portionwise and stirred for 2 h. The reaction was diluted with water and extracted with DCM (×3). The combined organic layers were washed (brine), dried (MgSO₄) then concentrated under reduced pressure to give the sub-title compound, yield 190 mg.

MS: ESI(+ve) 479 (M+H)

### (ii) 3-[4-chloro-2-({(3S)-3-methyl-4-[(3-methylbenzyl)sulfonyl]piperazin-1-yl}methyl)phenyl]propanoic acid

The title compound was prepared by the method of example 1 step (viii) using the product of step (i).

¹H NMR CD₃OD: δ 7.40 (1H, t), 7.42-7.38 (2H, m), 7.3-7.2 (4H, m), 4.44-4.34 (3H, m), 4.19-4.09 (2H, m), 3.56 (1H, d), 3.43-3.27 (2H, m), 3.15 (1H, d), 3.03-2.89 (3H, m), 2.83 (2H, t), 2.78-2.75 (1H, m), 2.36 (3H, s), 1.3 (3H, d).

MS: ESI(+ve) 465 (M+H)

### EXAMPLE 7

### 3-[4-chloro-2-({(3S)-3-methyl-4-[(2-methylbenzyl)sulfonyl]piperazin-1-yl}methyl)phenyl]propanoic acid

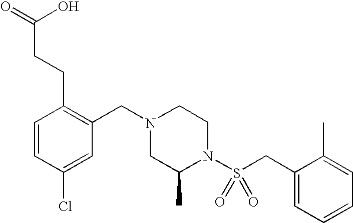

### (i) methyl 3-[4-chloro-2-({(3S)-3-methyl-4-[(2-methylbenzyl)sulfonyl]piperazin-1-yl}methyl)phenyl]propanoate

The sub-title compound was prepared by the method of example 5 step (vi) using the product of example 5 step (v) and (2-methylphenyl)methane sulfonyl chloride.

MS: ESI(+ve) 479 (M+H)

### (ii) 3-[4-chloro-2-({(3S)-3-methyl-4-[(2-methylbenzyl)sulfonyl]piperazin-1-yl}methyl)phenyl]propanoic acid

The title compound was prepared by the method of example 1 step (viii) using the product of step (i).

¹H NMR CD₃OD: δ 7.46 (1H, d), 7.44-7.34 (3H, m), 7.3-7.19 (3H, m), 4.45 (2H, s), 4.37 (1H, d), 4.21-4.09 (2H, m), 3.62 (1H, d), 3.45 (1H, td), 3.36-3.29 (1H, m), 3.18 (1H, d), 3.09 (1H, dd), 3.04-2.89 (3H, m), 2.83 (2H, t), 2.44 (3H, s), 1.34 (3H, d).

MS: APCI(+ve) 465 (M+H)

### EXAMPLE 8

### (2-{[(3S)-3-methyl-4-(phenylsulfonyl)piperazin-1-yl]methyl}phenyl)acetic acid

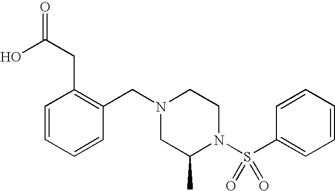

### (i) Ethyl[2-(bromomethyl)phenyl]acetate

Acetyl bromide (1 ml) was added dropwise to ethanol (10 ml) at 0° C. and stirred for 5 min. 3-isochromanone (0.56 g) was added and then allowed to reach RT and stirred for 16 h. The solvents were evaporated under reduced pressure to give the sub-title compound, yield 257 mg.

¹H NMR (CDCl₃) δ 7.40-7.20 (4H, m), 4.60 (2H, s), 4.16 (2H, q), 3.79 (2H, s), 1.26 (3H, t).

### (ii) (2-{[(3S)-3-methyl-4-(phenylsulfonyl)piperazin-1-yl]methyl}phenyl)acetic acid

Ethyl[2-(bromomethyl)phenyl]acetate (257 mg), the product of example 2 step (ii) (266 mg), ethanol (2 ml) and triethylamine (0.28 ml) were charged to a flask and heated at 60° C. for 4 h, then cooled to RT and the solvents were evaporated under reduced pressure. The reaction mixture was partitioned between EtOAc and water. The organic phase was dried (MgSO₄) then concentrated under reduced pressure. The residue was purified by SCX resin to give the ester. The ester was dissolved in a mixture of THF (2 ml) and 25% NaOH (1 ml), then stirred for 1 h at 57° C. The reaction mixture was cooled to RT, then acidified with acetic acid (10 ml) and then concentrated under reduced pressure. The residue was purified by RPHPLC to give the title compound as a white foam, yield 59 mg.

¹H NMR DMSO-D6: δ 7.79 (2H, m), 7.71-7.58 (3H, m), 7.24-7.13 (4H, m), 3.98 (1H, s), 3.72 (1H, d), 3.65 (1H, d), 3.52 (1H, d), 3.41 (1H, d), 3.33 (1H, m), 3.27 (1H, d), 3.05 (1H, dt), 2.56 (1H, m), 2.0 (1H, dd), 1.8 (1H, td), 1.0 (3H, d).

MS: APCI(−ve) 387 (M−H)

### EXAMPLE 9

### (4-chloro-2-{[(3S)-3-methyl-4-(phenylsulfonyl)piperazin-1-yl]methyl}phenyl)acetic acid

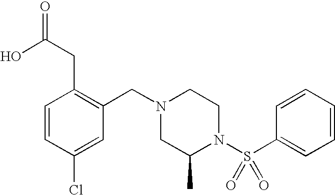

### (i) 4-chloro-1-(chloromethyl)-2-iodobenzene

Borane (24 ml, 1 M solution in THF) was added to a solution of 4-chloro-2-iodobenzoic acid (2.4 g) in THF (15 ml) and heated at 50° C. for 1 h, then cooled to RT. The reaction mixture was quenched with methanol and then concentrated under reduced pressure (2× azeotrope with methanol) to give a white solid. The solid was dissolved in DCM (20 ml) and DMF (1 ml) was added followed by dropwise addition of thionyl chloride (0.93 ml), then stirred for 1 h. The solvents were evaporated under reduced pressure and the residue was partitioned between diethyl ether and aqueous NaHCO₃. The organic phase was separated, dried (MgSO₄) then concentrated under reduced pressure to give the sub-title compound, yield 2.4 g. Used directly without characterisation.

### (ii) (4-chloro-2-iodophenyl)acetic acid

The product from step (i) (2.4 g) was dissolved in DMF (8 ml). Sodium cyanide (0.81 g) was added and the reaction mixture was stirred for 3 h at RT. Ice was added and a solid formed, which was filtered. The solid was dissolved in aqueous KOH (2.65 g in 14 ml water) and heated at 100° C. for 24 h, then allowed to cool to RT. The reaction mixture was washed with ether, then acidified and extracted with EtOAc (×2). The combined organic extracts were dried (Na₂SO₄) then concentrated under reduced pressure to give the sub-title compound as a yellow solid 1.93 g.

¹H NMR (CDCl₃) δ 7.85 (1H, d), 7.32 (1H, dd), 7.22 (1H, d), 3.83 (2H, s).

### (iii) methyl (4-chloro-2-iodophenyl)acetate

Trimethylsilyl chloride (2 ml) was added to a solution of the product from step (ii) (1.93 g) in MeOH (50 ml) and then stirred for 48 h. The solvent was evaporated under reduced pressure and the residue was purified by chromatography on silica (eluent diethyl ether) to give the sub-title compound as a yellow oil, yield 1.93 g

¹H NMR CDCl₃: δ 7.84 (1H, d), 7.31 (1H, dd), 7.21 (1H, d), 3.78 (2H, s), 3.72 (3H, s).

### (iv) methyl (4-chloro-2-vinylphenyl)acetate

The product from step (iii) (1.94 g), vinyltributyltin (2.19 ml), tetrakispalladium triphenylphosphine (0) (0.36 g) and toluene (10 ml) were charged to a flask and heated at 85° C. for 1 h, then at 110° C. for 16 h. The reaction mixture was allowed to cool to RT and the solvents evaporated under reduced pressure. The residue was purified by chromatography on silica (eluent 0-5% diethyl ether:hexane) to give the sub-title compound as a yellow oil, yield 1.05 g

¹H NMR CDCl₃: δ 7.48 (1H, d), 7.21 (1H, dd), 7.14 (1H, d), 6.86 (1H, dd), 5.66 (1H, dd), 5.39 (1H, dd), 3.68 (3H, s), 3.66 (2H, s).

### (v) methyl (4-chloro-2-formylphenyl)acetate

N-methyl-morpholine N-oxide (0.7 g) and osmium tetroxide (3 ml, 50% solution in water) were added to a mixture of the product from step (iv) (1.05 g) in tertiary butanol (29 ml), THF (9.7 ml) and water (2.9 ml). The reaction was stirred for 1 h then poured into saturated aq. NaHCO₃ (50 ml) and extracted with ether (×3). The combined organic extracts were dried (MgSO₄) then concentrated under reduced pressure to give the sub-title compound as a yellow oil, yield 0.71 g.

¹H NMR CDCl₃: δ 10.07 (1H, s), 7.82 (1H, d), 7.53 (1H, dd), 7.26 (1H, d), 4.02 (2H, s), 3.71 (3H, s).

### (vi) methyl (4-chloro-2-{[(3S)-3-methyl-4-(phenylsulfonyl)piperazin-1-yl]methyl}phenyl)acetate

The product of step (v) (200 mg), the product of example 3 step (ii) (330 mg), MgSO₄ (0.54 g) and anhydrous THF (3 ml) were charged to a flask and stirred for 6 h. Sodium triacetoxy borohydride (0.57 g) was added portionwise and the mixture was stirred for 16 h, then partitioned between 2 M Na₂CO₃ and EtOAc. The organic extracts were dried (Na₂SO₄) and concentrated under reduced pressure. The residue was purified by SCX (eluenting with MeCN, MeOH then 7% NH₃ in meOH). The product containing fractions were combined and then purified by chromatography on silica (1:1 diethyl ether:hexane) to give the sub-title compound as a colourless oil, yield 114 mg.

¹H NMR CDCl₃: δ 7.81 (2H, d), 7.54 (3H, m), 7.21 (2H, m), 7.13 (1H, d), 4.07 (1H, m), 3.79 (1H, d), 3.32 (1H, d), 3.32 (1H, d), 3.17 (1H, td), 2.63 (1H, d), 2.49 (1H, d), 2.18 (1H, dd), 2.0 (1H, td), 1.11 (3H, d).

### (vii) (4-chloro-2-{[(3S)-3-methyl-4-(phenylsulfonyl)piperazin-1-yl]methyl}phenyl)acetic acid

The title compound was prepared by the method of example 1 step (viii) using the product of step (vi).

¹H NMR DMSO-D6: δ 7.79 (2H, d), 7.68 (1H, tt), 7.61 (2H, m), 7.28 (1H, s), 7.27 (1H, dd), 7.22 (1H, d), 3.98 (1H, m), 3.70 (1H, d), 3.65 (1H, d), 3.53 (1H, d), 3.32 (2H, m), 3.06 (1H, dt), 2.56 (1H, d), 2.48 (1H, d), 2.00 (1H, dd), 1.83 (1H, dt), 1.01 (3H, t).

### EXAMPLES 10

### {4-chloro-2-[((3S)-3-methyl-4-{[4-(trifluoromethyl)phenyl]acetyl}piperazin-1-yl)methyl]phenyl}acetic acid

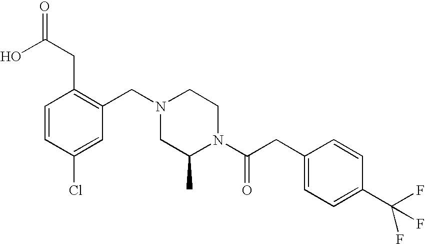

### (i) 5-chloro-2-(2-methoxy-2-oxoethyl)benzoic acid

Sodium formate (0.66 g), diisopropylethyl amine (1.12 ml), acetic anhydride (0.61 ml), and DMF (3.8 ml) were charged to a flask and stirred for 1 h. A solution of the product from example 9 step (iii) (1 g), Pd₂ dba₃ (75 mg) and lithium chloride (412 mg) in DMF (7.6 ml) was added and the reaction was stirred at 80° C. for 16 h. The reaction mixture was cooled to RT, then diluted with EtOAc and washed with 2M HCl (×3). The EtOAc layer was dried (Na₂SO₄) then concentrated under reduced pressure. The residue was purified by chromatography on silica (eluent EtOAc) to give the sub-title compound as a yellow oil, yield 398 mg

¹H NMR DMSO-D6: δ 7.88 (1H, d), 7.62 (1H, dd), 7.41 (1H, d), 4.01 (2H, s), 3.58 (3H, s).

### (ii) methyl[4-chloro-2-(hydroxymethyl)phenyl]acetate

Borane (1.7 ml, 1 M solution in THF) was added dropwise to a solution of the product of step (i) (398 mg) in THF (5 ml) at −0° C., then allowed to reach RT over 2 h. The reaction mixture was quenched with water, acidified to pH 3 and extracted with EtOAc (×3). The combined organic extracts were dried (Na₂SO₄) then concentrated under reduced pressure. The residue was purified by chromatography on silica (eluent EtOAc) to give the sub-title compound as a yellow oil, yield 335 mg

¹H NMR CDCl₃: δ 7.43 (1H, d), 7.25 (1H, dd), 7.17 (1H, d), 4.65 (2H, s), 3.72 (2H, s), 3.71 (3H, s).

### (iii) tert-butyl (2S)-4-[5-chloro-2-(2-methoxy-2-oxoethyl)benzyl]-2-methyl piperazine-1-carboxylate

Methanesulfonyl chloride (1.81 ml) was added to a solution of the product of step (ii) (2.85 g), triethylamine (3.72 ml) in DCM (15 ml) at 0° C. The reaction was stirred for 1 h at RT, then diluted with. The organic phase was washed with water, dried (Na₂SO₄) then concentrated under reduced pressure. The residue was purified by chromatography on silica (eluent 1:1 ether/isohexane) to give mesylate as a yellow oil. This mesylate was dissolved in DMF (7 ml) and K₂CO₃ (0.94 g) followed by the product of example 5 step (iii) (1.37 g) and heated at 75° C. for 4 h. The reaction mixture was allowed to cool to RT, and partitioned between EtOAc and water. The organic layer was washed with water, dried (Na₂SO₄) then concentrated under reduced pressure. The residue was purified by chromatography on silica (eluent 3:7 then 1:1 ether/isohexane) to give the sub-title compound as a yellow oil, yield 1.51 g.

¹H NMR DMSO-D6: δ 7.37 (1H, d), 7.33 (1H, dd), 7.27 (1H, d), 4.09 (1H, m), 3.87 (1.4H, s), 3.67 (1H, d), 3.62 (3H, s), 3.45 (1H, d), 3.35 (1H, d), 3.33 (0.6H, s), 2.89 (1H, dt), 2.58 (2H, m), 2.08 (1H, dd), 1.87 (1H, dt), 1.40 (9H, s), 1.12 (3H, d).

### (iv) methyl (4-chloro-2-{[(3S)-3-methylpiperazin-1-yl]methyl}phenyl)acetate TFA salt

TFA (10 ml) was added to a solution of the product of step (iii) (1.51 g) in DCM (2 ml) and stirred for 2 h, then concentrated under reduced pressure to give the sub-title compound as an oil, yield-quantitative.

MS: ESI(+ve) 297 (M+H)

### (v) {4-chloro-2-[((3S)-3-methyl-4-{[4-(trifluoromethyl)phenyl]acetyl}piperazin-1-yl)methyl]phenyl}acetic acid

DMF (1 drop) was added to a solution of oxalyl chloride (2 equivalents), [4-(trifluoromethyl)phenyl]acetic acid (0.14 g) in DCM and stirred for 1 h, then the solvents were removed under reduced pressure. The residue was dissolved in DCM (1 ml) and added dropwise to a vigorously stirred solution of the product of step (iv) (0.25 g), DCM (3 ml) and 3M aqueous K₂CO₃ (2 ml). The reaction was stirred for 2 days, then diluted with DCM (3 ml) and water. The organic phase was separated, washed (1M NaOH) and then concentrated under reduced pressure [MS: ESI(+ve) 483 (M+H)]. The residue was dissolved in THF (1 ml). 4N NaOH (1 ml) was added and the mixture was stirred vigorously for 4 h, then cooled to 0° C. and acidified with concentrated HCl (0.6 ml). The product was extracted with EtOAc and the organic phase was concentrated under reduced pressure, then purified by RPHPLC to give the title compound was a white solid.

¹H NMR DMSO-D6: δ 7.66 (2H, d), 7.47 (2H, d), 7.36 (1H, s), 7.27 (2H, m), 4.43 (1H, s), 3.96 (1H, s), 3.85 (1H, d), 3.79 (1H, d), 3.76 (1H, d), 3.70 (1H, d), 3.52 (1H, s), 3.44 (1H, d), 3.08 (1H, m), 2.73 (1H, d), 2.63 (1H, td), 2.14 (1H, dd), 1.95 (1H, td), 1.20 (3H, d).

MS: APCI(−ve) 435 (M−H)

Examples 11-14 were synthesised by the method of example 10 step (v) using the product of example 10 step (v) and the appropriate acid or sulfonyl chloride.

### EXAMPLE 11

### [4-chloro-2-({(3S)-4-[(4-methoxyphenyl)acetyl]-3-methylpiperazin-1-yl}methyl)phenyl]acetic acid

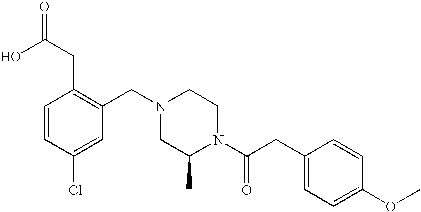

¹H NMR DMSO-D6: δ 7.36 (1H, s), 7.27 (2H, s), 7.16 (2H, d), 6.88 (2H, d), 4.41 (1H, s), 3.95 (1H, s), 3.77 (3H, s), 3.74 (2H, s), 3.65 (1H, d), 3.59 (1H, d), 3.48 (1H, d), 3.42 (1H, d), 3.02 (1H, t), 2.70 (1H, d), 2.61 (1H, d), 2.10 (1H, d), 1.91 (1H, t), 1.16 (3H, d).

MS: APCI(−ve) 429 (M−H)

### EXAMPLE 12

### [4-chloro-2-({(3S)-4-[(2,4-difluorophenyl)acetyl]-3-methylpiperazin-1-yl}methyl)phenyl]acetic acid

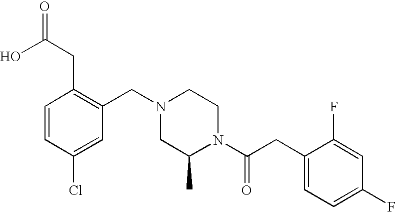

¹H NMR DMSO-D6: δ 7.38 (1H, s), 7.36-7.26 (3H, m), 7.09 (1H, dt), 7.00 (1H, tdd), 4.41 (1H, s), 3.96 (1H, m), 3.79 (1H, d), 3.73 (1H, d), 3.69 (2H, s), 3.51 (1H, d), 3.45 (1H, d), 3.10 (1H, m), 2.74 (1H, d), 2.65 (1H, td), 2.18 (1H, dd), 1.99 (1H, td), 1.23 (3H, d).

MS: APCI(−ve) 435 (M−H)

### EXAMPLE 13

### [4-chloro-2-({(3S)-4-[(3,4-difluorophenyl)acetyl]-3-methylpiperazin-1-yl}methyl)phenyl]acetic acid

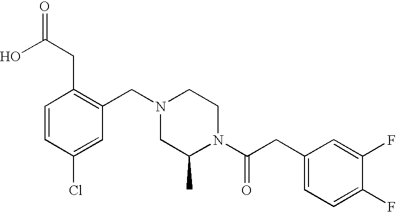

¹H NMR DMSO-D6: δ 7.37 (1H, s), 7.33-7.22 (4H, m), 7.08 (1H, m), 4.41 (1H, s), 3.96 (1H, s), 3.78 (1H, d), 3.72 (1H, d), 3.74 (1H, d), 3.68 (1H, d), 3.49 (1H, d), 3.43 (1H, d), 3.05 (1H, t), 2.72 (1H, d), 2.63 (1H, td), 2.13 (1H, dd), 1.94 (1H, td), 1.19 (3H, d).

MS: APCI(−ve) 435 (M−H)

### EXAMPLE 14

### (2-{[(3S)-4-(benzylsulfonyl)-3-methylpiperazin-1-yl]methyl}-4-chlorophenyl)acetic acid

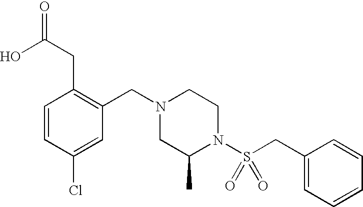

¹H NMR DMSO-D6: δ 7.46-7.33 (6H, m), 7.27 (2H, m), 4.39 (1H, d), 4.33 (1H, d), 3.83 (1H, m), 3.76 (1H, d), 3.70 (1H, d), 3.47 (1H, d), 3.42 (1H, d), 3.32 (1H, dt), 3.13 (1H, td), 2.60 (1H, d), 2.51 (1H, m), 2.17 (1H, dd), 2.00 (1H, td), 1.22 (3H, d).

MS: APCI(−ve) 435 (M−H)

### EXAMPLE 15

### [4-chloro-2-({(3S)-4-[(4-chlorophenyl)acetyl]-3-methylpiperazin-1-yl}methyl)phenyl]acetic acid

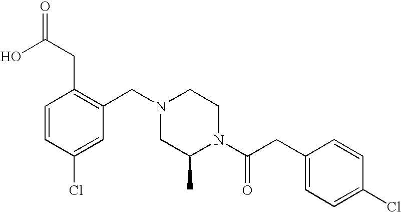

### (i) tert-butyl (3S)-4-[(4-chlorophenyl)acetyl]-3-methylpiperazine-1-carboxylate

The sub-title compound was prepared by the method of example 4 step (i) using the product of example 2 step (i) and (4-chloro)phenylacetyl chloride.

¹H NMR CDCl₃: δ 7.3 (2H, d), 7.22-7.13 (2H, m), 4.85-4.36 (1H, m), 4.08-3.15 (6H, m), 3.01-2.55 (2H, m), 1.45 (9H, s), 1.13 (3H, d).

### (ii) [4-chloro-2-({(3S)-4-[(4-chlorophenyl)acetyl]-3-methylpiperazin-1-yl}methyl)phenyl]acetic acid

The mesylate from example 10 step (ii) (300 mg), the product of step (i) (275 mg), K₂CO₃ (256 mg) and DMF (3 ml) were charged to a flask, then heated at 60° C. for 3 h. The reaction was allowed to cool to RT and partitioned between EtOAc and water. The organic layer was separated, washed with brine, dried (MgSO₄) and concentrated under reduced pressure. The residue was purified by SCX (eluent EtOAc, MeCN, MeOH then NH₃ in MeOH). The product containing fractions were concentrated under reduced pressure and the residue was purified by chromatography on silica (eluent ether) to give the sub-title compound as a yellow oil, yield 1.51 g.

MS: ESI(+ve) 449 (M+H)

### (iii) [4-chloro-2-({(3S)-4-[(4-chlorophenyl)acetyl]-3-methylpiperazin-1-yl}methyl)phenyl]acetic acid

The product from step (ii) was dissolved in a mixture of THF (3 ml) and 25% NaOH (3 ml), then stirred for 1 h at 50° C. The reaction mixture was cooled to RT, acidified with acetic acid (10 ml) and then concentrated under reduced pressure. The residue was purified by RPHPLC to give the title compound, yield 90 mg.

¹H NMR DMSO-D6: δ 7.31 (3H, m), 7.24 (4H, m), 4.37 (1H, s), 3.90 (1H, s), 3.72 (1H, d), 3.69 (1H, d), 3.66 (1H, d), 3.63 (1H, d), 3.45 (1H, d), 3.39 (1H, d), 3.01 (1H, m), 2.67 (1H, d), 2.58 (1H, d), 2.08 (1H, dd), 1.89 (1H, td), 1.14 (3H, d).

MS: APCI(−ve) 433 (M−H)

### EXAMPLE 16

### (4-chloro-2-{[(3S)-3-methyl-4-(phenylacetyl)piperazin-1-yl]methyl}phenyl)acetic acid

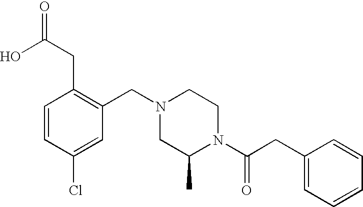

### (i) methyl (4-chloro-2-{[(3S)-3-methyl-4-(phenylacetyl)piperazin-1-yl]methyl}phenyl)acetate

HATU (0.28 g) was added to a stirred solution of the product of example 10 step (iv) (100 mg), phenyl acetic acid (102 mg), hunigs base (0.26 ml), DCM (2 ml) and NMP (2 ml). The reaction was stirred for 2 h, then diluted with water, extracted with EtOAc (×2). The combined organic extracts were washed with aqueous NaHCO₃, dried (Na₂SO₄) and then concentrated under reduced pressure. The residue was purified by chromatography on silica (eluent 8:2 ether/isohexane) to give the sub-title compound—used crude

MS: ESI(+ve) 415 (M+H)

### (ii) (4-chloro-2-{[(3S)-3-methyl-4-(phenylacetyl)piperazin-1-yl]methyl}phenyl)acetic acid

The title compound was prepared by the method of example 1 (viii) using the product of step (i).

¹H NMR DMSO-D6: δ 7.30-7.16 (8H, m), 4.38 (1H, s), 4.05-3.38 (7H, m), 3.01 (1H, s), 2.68 (1H, d), 2.57 (1H, d), 2.06 (1H, dd), 1.89 (1H, m), 1.13 (3H, d).

MS: APCI(−ve) 401 (M−H)

### EXAMPLE 17

### [4-chloro-2-({(3S)-4-[(4-fluorophenyl)acetyl]-3-methylpiperazin-1-yl}methyl)phenyl]acetic acid

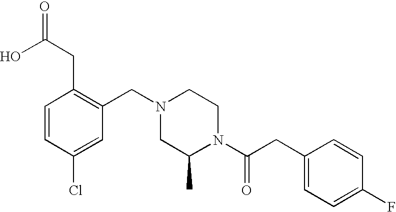

### (i) methyl[4-chloro-2-({(3S)-4-[(4-fluorophenyl)acetyl]-3-methylpiperazin-1-yl}methyl)phenyl]acetate

The sub-title compound was prepared by the method of example 16 step (i) using the product of example 10 step (iv).

### (ii) [4-chloro-2-({(3S)-4-[(4-fluorophenyl)acetyl]-3-methylpiperazin-1-yl}methyl)phenyl]acetic acid

The title compound was prepared by the method of example 1 (viii) using the product of step (i).

¹H NMR DMSO-D6: δ 7.29-6.95 (7H, m), 4.36 (1H, s), 4.03-3.22 (7H, m), 3.04 (1H, s), 2.71 (1H, d), 2.59 (1H, d), 2.06 (1H, dd), 1.90 (1H, td), 1.15 (3H, d).

MS: APCI(−ve) 419 (M−H)

### EXAMPLE 18

### [4-chloro-2-({(3S)-3-ethyl-4-[(4-fluorophenyl)acetyl]piperazin-1-yl}methyl)phenyl]acetic acid

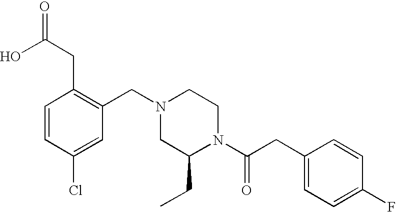

### i) (3S)-3-Ethyl-1-(phenylmethyl)-2,5-piperazinedione

To a solution of DCC (5.07 g) in DCM (140 ml) at 0° C. was added N—BOC-L-α-aminobutyric acid (5 g) followed by ethyl N-benzylglycinate (4.6 mL) dropwise. The resulting solution was stirred at 0° C. for 2 h and then at RT 1 h, filtered and the concentrated to give an oil. This was dissolved in DCM (100 mL) and TFA (100 ml) and stirred for 1 h. The solution was concentrated under reduced pressure. The residue was stirred in saturated aq NaHCO₃ (125 ml) and EtOAc (125 ml) for 6 h. The organics were separated, dried (Na₂SO₄), and concentrated to give the sub-title compound as a white solid. (5.68 g).

¹H NMR (CDCl₃) δ 7.37-7.31 (3H, m), 7.26 (2H, m), 6.80 (1H, s), 4.70 (1H, d), 4.50 (1H, d), 4.05 (1H, s), 3.87 (1H, d), 3.80 (1H, d), 1.93 (2H, m), 0.98 (3H, t).

### ii) (3S)-3-Ethyl-1-(phenylmethyl)-piperazine

To a solution of the product of example 69 part a) (5.68 g) in THF (30 ml) at 0° C. was added LAH (100 ml, 1.0M in THF) dropwise. The resulting solution was heated at reflux overnight. The reaction mixture was cooled to RT and quenched by cautious sequential addition of water (3.8 ml), 15% aq NaOH (3.8 ml), and water (11.4 ml). The precipitous solution was diluted with EtOAc and filtered through Celite. The residue was washed with EtOAc (3×100 ml) and the combined organics concentrated in vacuo. The crude product was dissolved in DCM, filtered through Celite and the solvent removed in vacuo to give the sub-title product as a yellow oil (4.74 g).

¹H NMR (CDCl₃) δ 7.41-7.19 (5H, m), 3.53 (1H, d), 3.46 (1H, d), 2.99-2.61 (5H, m), 2.01 (1H, dt), 1.69 (1H, t), 1.35 (2H, dquin), 0.90 (3H, t).

### iii) (2S)-2-Ethyl-4-(phenylmethyl)-1-piperazinecarboxylic acid, 1,1-dimethylethyl ester

To a solution of the product from example 69 part b) (4.74 g) in DCM (150 ml) was added (BOC)₂O (5.52 g) and the reaction stirred at RT for 48 h. The reaction was concentrated under reduced pressure. The crude product was purified by chromatography (silica, (0-10% EtOAc/isohexane as eluent)), to give the sub-titled compound as a colourless oil (6.09 g).

¹H NMR (CDCl₃) δ 7.33-7.22 (5H, m), 3.89 (2H, m), 3.53 (1H, d), 3.38 (1H, d), 3.04 (1H, t), 2.71 (2H, dd), 2.02 (2H, ddd), 1.83 (1H, m), 1.64 (1H, m), 1.45 (9H, s), 0.80 (3H, t).

### iv) (2S)-2-Ethyl-1-piperazinecarboxylic acid, 1,1-dimethylethyl ester

A solution of the product from example 69 part c) (6.09 g) and 10% Pd/C (1.14 g) in EtOH (85 mL) was hydrogenated at 3.8 bar for 16 h. The reaction mixture was filtered through Celite and the filtrate concentrated in vacuo to give the sub-title compound as an oil (3.65 g).

¹H NMR (CDCl₃) δ 3.87 (2H, m), 2.87 (4H, m), 2.68 (1H, td), 1.76 (1H, m), 1.59 (1H, m), 1.46 (9H, s), 0.89 (3H, t).

### v) tert-butyl (2S)-4-[5-chloro-2-(2-methoxy-2-oxoethyl)benzyl]-2-ethylpiperazine-1-carboxylate

The sub-title compound was prepared by the method of example 10 step (iii) using the products of example 10 step (ii) and the product of step (iv).

¹H NMR DMSO-D6: δ 7.33 (1H, d), 7.30 (1H, dd), 7.25 (1H, d), 3.83 (2H, s), 3.71 (1H, d), 3.60 (3H, s), 3.42 (1H, d), 3.31 (1H, m), 2.81 (2H, m), 2.63 (1H, d), 2.57 (1H, d), 1.99 (1H, dd), 1.85 (1H, td), 1.63 (1H, m), 1.53 (1H, m), 1.38 (9H, s), 0.73 (3H, t).

### vi) methyl (4-chloro-2-{[(3S)-3-ethylpiperazin-1-yl]methyl}phenyl)acetate trifluoroacetate

The sub-title compound was prepared by the method of example 10 step (iv) using the product of step (v).

### vii) [4-chloro-2-({(3S)-3-ethyl-4-[(4-fluorophenyl)acetyl]piperazin-1-yl}methyl)phenyl]acetic acid

The title compound was prepared by the method of example 2 step (ii) and the method of example 1 step (viii) using the product of step (vi) and 4-fluorophenylacetyl chloride.

¹H NMR DMSO-D6 (90° C.): δ 7.35 (1H, s), 7.27 (4H, m), 7.11 (2H, t), 4.06 (2H, m), 3.78-3.64 (4H, m), 3.47 (1H, d), 3.42 (1H, d), 3.00 (1H, s), 2.71 (2H, m), 2.02 (1H, dd), 1.92 (1H, td), 1.67 (2H, m), 0.74 (3H, t).

MS: APCI(−ve) 431 (M−H)

### EXAMPLE 19

### [4-chloro-2-({(3S)-4-[(4-chlorophenyl)acetyl]-3-ethylpiperazin-1-yl}methyl)phenyl]acetic acid

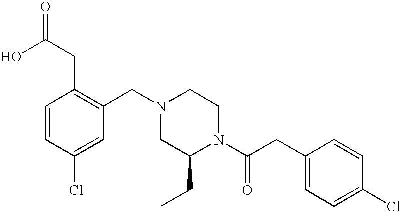

The title compound was prepared by the method of example 2 step (ii) and the method of example 1 step (viii) using the product of step example 18 step (vi) and 4-chlorophenylacetyl chloride.

¹H NMR DMSO-D6 (90° C.): δ 7.39-7.20 (7H, m), 3.89-2.84 (5H, m), 3.75 (1H, d), 3.67 (1H, d), 3.64 (1H, d), 3.58 (1H, d), 2.73 (2H, d), 2.02 (1H, dd), 1.96 (1H, dd), 1.69 (2H, m), 0.75 (3H, t).

MS: APCI(−ve) 447 (M−H)

### EXAMPLE 20

### 2-(2-{[(3S)-4-(benzylsulfonyl)-3-methylpiperazin-1-yl]methyl}-4-chlorophenyl)-N-(methylsulfonyl)acetamide

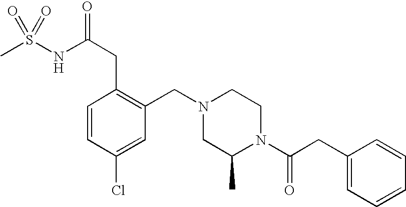

The product of example 15 step (iii) (50 mg) was taken up in DCM (1 ml) and methane sulfonamide (13 mg) and PyBOP (89 mg) added followed by Hunigs base (0.06 ml). The mixture was stirred at room temperature for 16 h then evaporated under reduced pressure and the residue purified by RPHPLC. The resulting fractions were evaporated under reduced pressure and passed through an SCX resin (eluting with methanol then 7N ammonia in methanol). The basic fractions were evaporated under reduced pressure to give a white solid (13 mg).

¹H NMR DMSO-D6: δ 7.42-7.34 (5H, m), 7.31 (1H, d), 7.24 (1H, dd), 7.19 (1H, d) 4.41 (1H, d), 4.35 (1H, d), 3.77 (1H, m), 3.62 (1H, d), 3.53 (1H, d), 3.41 (2H, s), 3.12 (2H, m), 2.94 (3H, s), 2.60 (1H, d), 2.46 (1H, d), 2.07 (1H, dd), 1.95 (1H, dt), 1.18 (3H, d).

MS: APCI(+ve) 514 (M+H).

### Pharmacological Data

### Ligand Binding Assay

[³H]PGD₂ was purchased from Perkin Elmer Life Sciences with a specific activity of 100-210 Ci/mmol. All other chemicals were of analytical grade.

HEK cells expressing rhCRTh2/Gα16 were routinely maintained in DMEM containing 10% Foetal Bovine Serum (HyClone), 1 mg/ml geneticin, 2 mM L-glutamine and 1% non-essential amino acids. For the preparation of membranes, the adherent transfected HEK cells were grown to confluence in two layer tissue culture factories (Fisher, catalogue number TKT-170-070E). Maximal levels of receptor expression were induced by addition of 500 mM sodium butyrate for the last 18 h of culture. The adherent cells were washed once with phosphate buffered saline (PBS, 50 ml per cell factory) and detached by the addition of 50 ml per cell factory of ice-cold membrane homogenisation buffer [20 mM HEPES (pH 7.4), 0.1 mM dithiothreitol, 1 mM EDTA, 0.1 mM phenyl methyl sulphonyl fluoride and 100 μg/ml bacitracin]. Cells were pelleted by centrifugation at 220×g for 10 minutes at 4° C., re-suspended in half the original volume of fresh membrane homogenisation buffer and disrupted using a Polytron homogeniser for 2×20 second bursts keeping the tube in ice at all times. Unbroken cells were removed by centrifugation at 220×g for 10 minutes at 4° C. and the membrane fraction pelleted by centrifugation at 90000×g for 30 minutes at 4° C. The final pellet was re-suspended in 4 ml of membrane homogenisation buffer per cell factory used and the protein content determined. Membranes were stored at −80° C. in suitable aliquots.

All assays were performed in Corning clear bottomed, white 96-well NBS plates (Fisher). Prior to assay, the HEK cells membranes containing CRTh2 were coated onto SPA PVT WGA beads (Amersham). For coating membranes were incubated with beads at typically 25 μg membrane protein per mg beads at 4° C. with constant agitation overnight. (The optimum coating concentrations were determined for each batch of membranes) The beads were pelleted by centrifugation (800×g for 7 minutes at 4° C.), washed once with assay buffer (50 mM HEPES pH 7.4 containing 5 mM magnesium chloride) and finally re-suspended in assay buffer at a bead concentration of 10 mg/ml.

Each assay contained 20 μl of 6.25 nM [³H]PGD₂, 20 μl membrane saturated SPA beads both in assay buffer and 10 μl of compound solution or 13,14-dihydro-15-keto prostaglandin D₂ (DK-PGD₂, for determination of non-specific binding, Cayman chemical company). Compounds and DK-PGD₂ were dissolved in DMSO and diluted in the same solvent to 100× the required final concentration. Assay buffer was added to give a final concentration of 10% DMSO (compounds were now at 10× the required final concentration) and this was the solution added to the assay plate. The assay plate was incubated at RT for 2 h and counted on a Wallac Microbeta liquid scintillation counter (1 minute per well).

Compounds of formula (I) have an IC₅₀ value of less than (<) 10 μM. Specifically Example 4 has a pIC₅₀ value of 7.1, example 9 has a pIC₅₀ value of 7.85, example 12 has a pIC₅₀ value of 8.1.

1. A compound of formula (I) or a carboxylic acid bioisostere thereof: 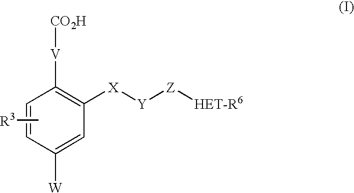
in which:
V is CR¹R², CR¹R²—CR¹R² or V is S(O)ₙCR¹R² (where n is 0, 1 or 2), NR¹¹CR¹R², CCR¹R², CR¹R²C or CR¹CR²;
R¹ and R² independently represent a hydrogen atom, halogen, C₂-C₆ alkenyl, C₂-C₆ alkynyl, C₃-C₇ cycloalkyl or a C₁₋₆alkyl group, the latter four groups being optionally substituted by one or more substituents independently selected from halogen, C₃-C₇ cycloalkyl, NR⁹R¹⁰, OR⁸, S(O)ₙR⁷ (where n is 0, 1 or 2);
or
R¹ and R² together can form a 3-8 membered ring optionally containing one or more atoms selected from O, S, NR¹¹ and itself optionally substituted by one or more C₁-C₃ alkyl or halogen;
W is hydrogen, halogen, cyano, nitro, SO₂R⁷, SO₂NR⁹R¹⁰, OR⁸, or C₁₋₆alkyl, the latter being optionally substituted by one or more substituents independently selected from halogen, OR⁸ and NR⁷R⁸, S(O)ₙR⁵, where n is 0, 1 or 2.
R³ is one or more substituents independently selected from hydrogen, halogen, CN, nitro, SO₂R⁷, OR⁸, SR⁷, SOR⁷, SO₂NR⁹R¹⁰, CONR⁹R¹⁰, NR⁹R¹⁰, NR¹¹SO₂R⁷, NR¹¹CO₂R⁷, NR¹¹COR⁷ or C₁₋₆alkyl, the latter being optionally substituted by one or more substituents independently selected from halogen, OR⁸ and NR⁹R¹⁰, S(O)ₙR⁷ where n is 0, 1 or 2;
X represents a bond, or C₁-C₆ alkyl, optionally substituted by one or more substituents independently selected from halogen, C₁-C₆ alkyl the latter being optionally substituted by one or more substituents independently selected from halogen, OR⁶ and NR⁷R⁸, S(O)ₙR⁵ where n is 0, 1 or 2;
Y represents a diamine of the following type:— 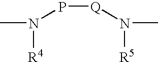
R⁴ and R⁵ independently represent hydrogen, SO₂R⁷, C(O)R⁷, CO₂R⁷ and C₁-C₆ alkyl, the latter being optionally substituted by one or more substituents independently selected from aryl, heteroaryl, halogen, OR⁸ and NR⁹R¹⁰, S(O)ₙR⁷ where n is 0, 1 or 2;
R⁴ and R⁵ are joined together or one of R⁴ and R⁵ is joined onto P or Q to form a saturated heterocyclic 3-10 membered ring with, 1 or 2 endocyclic nitrogen atoms;
P and Q independently represent, C₁-C₆ alkyl optionally substituted by one or more substituents independently selected from (═O), halogen, OR⁸ and NR⁹R¹⁰, S(O)ₙR⁷ (where n is 0, 1 or 2), C₁-C₆ alkyl, C₃-C₆ cycloalkyl, aryl or heteroaryl (the latter two being optionally substituted by one or more substituents independently selected from halogen, OR⁸ and NR⁹R¹⁰, CONR⁹R¹⁰, S(O)ₙR⁷ where n is 0, 1 or 2);
Z represents a bond, (CR¹²)n-C(O), (CR¹²)n-S(O)n, C(O)(CR¹²)n, or S(O)₂(CR¹²)n, S(O)₂N(CR¹²)n, where n=0, 1 or 2;
HET represents aryl or heteroaryl;
R⁶ represents one or more substituents independently selected from hydrogen, halogen, CN, nitro, COR⁷, CO₂R⁸, SO₂R⁷, OR⁸, SR⁸, SOR⁷, SO₂NR⁹R¹⁰, CONR⁹R¹⁰, NR⁹R¹⁰, NR⁸SO₂R⁷, NR⁸CO₂R⁸, NR⁸COR⁷, NR⁸CONR⁹R¹⁰, NR⁸SO₂NR⁹R¹⁰, aryl, heteroaryl, C₂-C₆ alkenyl, C₂-C₆ alkynyl, C₃-C₇ cycloalkyl or C₁₋₆alkyl, the latter four groups being optionally substituted by one or more substituents independently selected from halogen, C₃-C₇ cycloalkyl, CN, OR⁸, NR⁹R¹⁰, S(O)ₙR⁷ (where n is 0, 1 or 2), CONR⁹R¹⁰, NR⁸COR⁷, SO₂NR⁹R¹⁰ and NR⁸SO₂R⁷;
R⁷ represents a C₁-C₆ alkyl, an aryl or a heteroaryl group all of which may be optionally substituted by halogen atoms, OR⁸, NR¹⁴R¹⁵;
R⁸ represents hydrogen, C₁-C₆, alkyl, an aryl or a heteroaryl group all of which may be optionally substituted by halogen atoms, OR⁸, NR¹⁴R¹⁵;
R⁹ and R¹⁰ independently represent hydrogen, C₃-C₇ cycloalkyl or C₁₋₆alkyl, the latter two groups being optionally substituted by one or more substituents independently selected from halogen, C₃-C₇ cycloalkyl, OR⁶ and NR¹⁴R¹⁵, S(O)ₙR⁶ (where n=0, 1 or 2), CONR⁷R⁸, NR⁶COR⁷, SO₂NR⁷R⁸ and NR⁶SO₂R⁵;
or
R⁹ and R¹⁰ together with the nitrogen atom to which they are attached can form a 3-8 membered saturated heterocylic ring optionally containing one or more atoms selected from O, S(O)ₙ (where n=0, 1 or 2), NR¹³, and itself optionally substituted by halogen or C₁₋₃ alkyl;
R¹¹ represents a hydrogen atom, C(O)R⁹, C₁-C₆ alkyl an aryl or a heteroaryl group (the latter three can be optionally substituted by halogen);
R¹² represents one or more from hydrogen, or a C₁₋₆alkyl group, the latter being optionally substituted by one or more substituents independently selected from halogen, C₃-C₇ cycloalkyl, NR¹⁴R¹⁵, OR⁸, S(O)ₙR⁷ (where n is 0, 1 or 2);
R¹³ represent hydrogen, C₁₋₄ alkyl, —COC₁-C₄ alkyl, COYC₁-C₄alkyl where Y is O or NR⁷; and
R¹⁴ and R¹⁵ independently represent hydrogen, C₁₋₄ alkyl
or
R¹⁴ and R¹⁵ together with the nitrogen atom to which they are attached can form a 3-8 membered saturated heterocylic ring optionally containing one or more atoms selected from O, S(O)ₙ (where n=0, 1 or 2), NR¹³, and itself optionally substituted by halogen or C₁₋₃ alkyl;
and pharmaceutically acceptable salts thereof.

2. A compound according to claim 1 in which V is CR¹R², CR¹R²—CR¹R², CCR¹R² or CR¹R²C.

3. A compound according to claim 1 or 2 in which W is hydrogen, halogen or CF₃.

4. A compound according to any one of claims 1 to 3 in which R¹ and R² are hydrogen.

5. A compound according to any one of claims 1 to 4 in which R³ is hydrogen.

6. A compound according to any one of claims 1 to 5 in which X is CH₂;

7. A compound according to any one of claims 1 to 6 in which the group Z is SO₂, SO₂CH₂, C(O)CH₂.

8. A compound according to any one of claims 1 to 7 in which the group Y together with the 2 nitrogen atoms it is attached forms a 4-7 membered saturated ring, optionally substituted by C₁₋₄ alkyl.

9. A compound according to any one of claims 1 to 8 in which the carboxylic acid bioisostere is a group of formula (XI) to (XV):

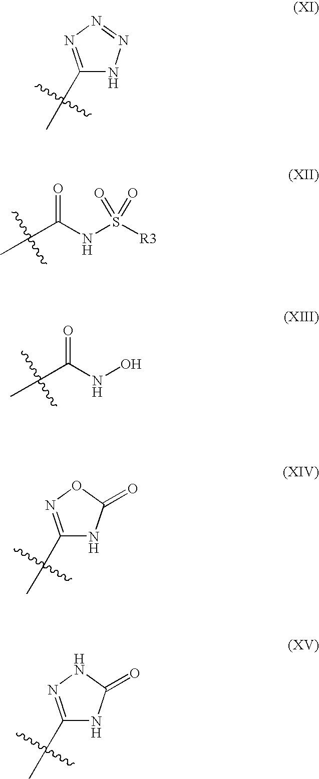

10. A compound of formula (I) according to any one of claims 1 to 5 selected from:
Sodium 3-(2-{[4-(benzylsulfonyl)piperazin-1-yl]methyl}-4-chlorophenyl) propanoate;
3-(2-{[(3S)-4-(benzylsulfonyl)-3-methylpiperazin-1-yl]methyl}-4-chlorophenyl)propanoic acid;
Sodium 3-(4-chloro-2-{[(3S)-3-methyl-4-(phenylsulfonyl)piperazin-1-yl]methyl}phenyl)propanoate;
3-(4-chloro-2-{[(3S)-3-methyl-4-(phenylacetyl)piperazin-1-yl]methyl}phenyl)propanoic acid;
3-[4-chloro-2-({(3S)-3-methyl-4-[(4-methylbenzyl)sulfonyl]piperazin-1-yl}methyl)phenyl]propanoic acid;
3-[4-chloro-2-({(3S)-3-methyl-4-[(3-methylbenzyl)sulfonyl]piperazin-1-yl}methyl)phenyl]propanoic acid;
3-[4-chloro-2-({(3S)-3-methyl-4-[(2-methylbenzyl)sulfonyl]piperazin-1-yl}methyl)phenyl]propanoic acid;
(2-{[(3S)-3-methyl-4-(phenylsulfonyl)piperazin-1-yl]methyl}phenyl)acetic acid;
(4-chloro-2-{[(3S)-3-methyl-4-(phenylsulfonyl)piperazin-1-yl]methyl}phenyl)acetic acid;
{4-chloro-2-[((3S)-3-methyl-4-{[4-(trifluoromethyl)phenyl]acetyl}piperazin-1-yl)methyl]phenyl}acetic acid;
[4-chloro-2-({(3S)-4-[(4-methoxyphenyl)acetyl]-3-methylpiperazin-1-yl}methyl)phenyl]acetic acid;
[4-chloro-2-({(3S)-4-[(2,4-difluorophenyl)acetyl]-3-methylpiperazin-1-yl}methyl)phenyl]acetic acid;
[4-chloro-2-({(3S)-4-[(3,4-difluorophenyl)acetyl]-3-methylpiperazin-1-yl}methyl)phenyl]acetic acid;
(2-{[(3S)-4-(benzylsulfonyl)-3-methylpiperazin-1-yl]methyl}-4-chlorophenyl)acetic acid;
[4-chloro-2-({(3S)-4-[(4-chlorophenyl)acetyl]-3-methylpiperazin-1-yl}methyl)phenyl]acetic acid;
(4-chloro-2-{[(3S)-3-methyl-4-(phenylacetyl)piperazin-1-yl]methyl}phenyl)acetic acid;
[4-chloro-2-({(3S)-4-[(4-fluorophenyl)acetyl]-3-methylpiperazin-1-yl}methyl)phenyl]acetic acid;
[4-chloro-2-({(3S)-3-ethyl-4-[(4-fluorophenyl)acetyl]piperazin-1-yl}methyl)phenyl]acetic acid;
[4-chloro-2-({(3S)-4-[(4-chlorophenyl)acetyl]-3-ethylpiperazin-1-yl}methyl)phenyl]acetic acid;
2-(2-{[(3S)-4-(benzylsulfonyl)-3-methylpiperazin-1-yl]methyl}-4-chlorophenyl)-N-(methylsulfonyl)acetamide and pharmaceutically acceptable salts thereof.

11. A compound of formula (I) according to any one of claims 1 to 10 for use in therapy.

12. A method of treating a disease mediated by prostaglandins, which comprises administering to a patient a therapeutically effective amount of a compound of formula (I), or a pharmaceutically acceptable salt as defined in claims 1 to 10.

13. A method of treating a disease mediated by prostaglandin D2, which comprises administering to a patient a therapeutically effective amount of a compound of formula (I), or a pharmaceutically acceptable salt as defined in claims 1 to 10.

14. A method of treating a respiratory disease, such as asthma and rhinitis, in a patient suffering from, or at risk of, said disease, which comprises administering to the patient a therapeutically effective amount of a compound of formula (I), or a pharmaceutically acceptable salt or solvate thereof, as defined in claims 1 to 10.
